(19) United States
(12) Patent Application Publication
(10) Pub. No.: US 2026/0224906 A1
(43) Pub. Date: Aug. 6, 2026

(54) MEDICAL DEVICE AND METHOD FOR DELIVERING CARDIAC PACING PULSES

(71) Applicant: Medtronic, Inc., Minneapolis, MN (US)

(72) Inventors: Marshall J. RASMUSSEN, Mesa, AZ (US); Anderson Kristopher SMITH, Phoenix, AZ (US); Benjamin Ryan ALMQUIST, Roseville, MN (US); Troy M. RELLER, Ham Lake, MN (US); Kevin P. KUEHN, Shoreview, MN (US); Randolph E. CRUTCHFIELD, Scottsdale, AZ (US); Timothy A. EBELING, Circle Pines, MN (US)

(21) Appl. No.: 19/150,895

(22) PCT Filed: Jan. 22, 2024
(86) PCT No.: PCT/IB2024/050588

§ 371 Date: Jul. 24, 2025

Related U.S. Application Data
(60) Provisional application No. 63/481,795, filed on Jan. 26, 2023.

(51) Int. Cl. A61N 1/39; A61N 1/02; A61N 1/05; A61N 1/378

(52) U.S. Cl. CPC A61N 1/39622; A61N 1/025; A61N 1/0597; A61N 1/378; A61N 1/3912

(57) ABSTRACT

A medical device is configured to deliver a cardiac pacing pulse having a pacing pulse amplitude for delivery to an electrode terminal of the medical device. The medical device is configured to pull a latching current by an internal adjustable load of the medical device coupled to a low side of a high side switch of a high voltage output circuit of the medical device to hold the high side switch in a conducting state during a first portion of the cardiac pacing pulse in some examples. The medical device may be configured to pull a holding current to hold the high side switch in a conducting state during a second portion of the cardiac pacing pulse. The holding current may be less than the latching current.

This application claims the benefit of U.S. Provisional Patent Application Ser. No. 63/481,795, filed Jan. 26, 2023, the entire content of which is incorporated herein by reference.

### TECHNICAL FIELD

The disclosure relates generally to a medical device and method for delivering cardiac pacing pulses.

### BACKGROUND

Medical devices may sense electrophysiological signals from the heart, brain, nerve, muscle or other tissue. Such devices may be implantable, wearable or external devices using implantable and/or surface (skin) electrodes for sensing the electrophysiological signals. In some cases, such devices may be configured to deliver a therapy based on the sensed electrophysiological signals. For example, implantable or external cardiac pacemakers, cardioverter defibrillators, cardiac monitors and the like, sense cardiac electrical signals from a patient's heart. The medical device may sense cardiac electrical signals from the heart and deliver electrical stimulation therapies, such as cardiac pacing pulses and/or cardioversion or defibrillation (CV/DF) shocks, to the heart using electrodes, which may be carried by medical electrical leads extending from the medical device to position electrodes within or near the patient's heart.

A cardiac pacemaker or cardioverter defibrillator may deliver therapeutic electrical stimulation to the heart via electrodes carried by one or more medical electrical leads coupled to the medical device. Cardiac signals sensed from the heart may be analyzed for detecting an abnormal rhythm. Upon detection of an abnormal rhythm, such as bradycardia, tachycardia or fibrillation, an appropriate electrical stimulation pulse or pulses may be delivered to restore or maintain a more normal rhythm of the heart. For example, an implantable cardioverter defibrillator (ICD) may deliver bradycardia pacing pulses to the heart of the patient in the absence of sensed intrinsic myocardial depolarization signals, e.g., R-waves, deliver anti-tachycardia pacing pulses in response to detecting tachycardia, or deliver CV/DF shocks to the heart upon detecting tachycardia or fibrillation.

### SUMMARY

In general, the disclosure is directed to a medical device and techniques for delivering cardiac pacing pulses. The cardiac pacing pulses may be delivered using high surface area, low impedance electrodes, which may not be in contact with a patient's heart in some examples. The medical device may be a pacemaker or ICD configured to deliver cardiac pacing pulses using extra-cardiac electrodes, e.g., electrodes carried by non-transvenous leads or transvenous leads positioned outside the heart, in an extra-cardiac location. A medical device operating according to the techniques disclosed herein may generate cardiac pacing pulses that are delivered to high surface area, low impedance pacing electrode vector via output circuit switching circuitry controlled in part using an internal adjustable load. The low impedance pacing electrode vector may include at least one high surface area electrode that can be used for delivering high voltage CV/DF shocks in some examples.

Control circuitry of the medical device may control the internal adjustable load to pull current through the output circuit switching circuitry. The current pulled by the internal adjustable load in combination with the current flowing through an external pacing load during a cardiac pacing pulse is controlled to be high enough to hold the switching circuitry in a conducting state for delivering the cardiac pacing pulse via the low impedance pacing electrode vector. In some examples, the control circuitry adjusts the internal adjustable load during the cardiac pacing pulse to pull a second current that is lower than the first current to hold the output circuit switching circuitry in the conducting state to complete delivery of a phase of the cardiac pacing pulse. For example, the internal adjustable load may pull a latching current during a first portion of the pacing pulse and a holding current that is less than the latching current during a second portion of the pacing pulse. The first and second portions of the pacing pulse may be during a given phase of the cardiac pacing pulse, which may be a monophasic pulse or a biphasic or other multiphasic pulse in various examples.

In one example, the disclosure provides a medical device including a therapy delivery circuit configured to deliver electrical stimulation pulses. The therapy delivery circuit can include a first electrode terminal, a second electrode terminal, a high voltage output circuit including a high side switch coupled to the first electrode terminal, an internal adjustable load coupled to a low side of the high side switch and a cardiac pacing voltage source configured to generate a cardiac pacing pulse having a pacing pulse amplitude. The medical device further includes control circuitry configured to control the therapy delivery circuit to deliver the cardiac pacing pulse via the first electrode terminal and the second electrode terminal by controlling the internal adjustable load to pull a latching current to hold the high side switch in a conducting state during a first portion of the cardiac pacing pulse. The control circuitry is further configured to control the internal adjustable load to pull a holding current to hold the high side switch in a conducting state during a second portion of the cardiac pacing pulse, the holding current being less than the latching current.

In another example, the disclosure provides a method including generating a cardiac pacing pulse having a pacing pulse amplitude for delivery via a first electrode terminal and a second electrode terminal of a medical device. The method may include pulling a latching current by an internal adjustable load of the medical device coupled to a low side of a high side switch coupled to the first electrode terminal to hold the high side switch in a conducting state during a first portion of the first cardiac pacing pulse. The method may further include pulling a holding current to hold the high side switch in a conducting state during a second portion of the cardiac pacing pulse, the holding current being less than the latching current.

In yet another example, the disclosure provides a non-transitory computer readable medium storing a set of instructions that, when executed by control circuitry of a medical device, cause the medical device to generate a cardiac pacing pulse having a pacing pulse amplitude for delivery to an electrode terminal of the medical device. The instructions may cause the medical device to pull a latching current by an internal adjustable load of the medical device coupled to a low side of a high side switch of a high voltage output circuit of the medical device to hold the high side switch in a conducting state during a first portion of the cardiac pacing pulse. The instructions may further cause the medical device to pull a holding current to hold the high side switch in a conducting state during a second portion of the cardiac pacing pulse, the holding current being less than the latching current.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIGS. 1A and 1B are conceptual diagrams of one example of an ICD system that may be configured to sense cardiac event signals and deliver electrical stimulation therapy according to the techniques disclosed herein.

FIGS. 2A-2C are conceptual diagrams of a patient implanted with an ICD system in a different implant configuration than the arrangement shown in FIGS. 1A-1B.

FIG. 3 is a conceptual diagram of an ICD according to some examples.

FIG. 4 is a conceptual diagram of circuitry that can be included in the therapy delivery circuit of FIG. 3 according to some examples.

FIG. 5 is a conceptual diagram of therapy delivery circuitry according to another example.

FIG. 6 is a diagram of a cardiac pacing pulse and the current that may be pulled by an internal adjustable load during the cardiac pacing pulse according to some examples.

FIG. 7 is a diagram of a biphasic cardiac pacing pulse and a corresponding current signal that may be drawn by an internal adjustable load according to another example.

FIG. 8 is a flow chart of a method for delivering cardiac pacing pulses by a medical device according to some examples.

FIG. 9 is a flow chart of a method for delivering cardiac pacing pulses by an ICD via the HV output circuit of FIG. 4 using an internal adjustable load according to another example.

### DETAILED DESCRIPTION

In general, this disclosure describes medical devices and techniques for delivering cardiac pacing pulses. The cardiac pacing pulses may be delivered using relatively high surface area, low impedance electrodes that may be implanted in an extra-cardiac or extra-cardiovascular location. The high surface area electrodes may be used for delivering CV/DF shocks by the medical device. When a high voltage CV/DF shock is delivered, a high voltage capacitor is discharged through a high voltage output circuit to a low impedance CV/DF shock electrode vector. High current conducted through the high voltage output circuit maintains charge coupled components of the high voltage output circuit in a conducting state for discharging the high voltage capacitor for shock delivery. At times, the high surface area, low impedance electrodes normally used for delivering CV/DF shock pulses may be needed for delivering cardiac pacing pulses to the patient's heart. Because the cardiac pacing pulses are generally much lower in voltage amplitude than a CV/DF shock pulse, the current through the high voltage output circuit is insufficient for holding the charge coupled components of the high voltage output circuit in a conducting state. Apparatus and techniques are disclosed herein for controlling an internal adjustable load to conduct relatively low voltage cardiac pacing pulses via the high voltage output circuitry to electrode terminals coupled to a low impedance pacing electrode vector.

As used herein, the term “extra-cardiac” refers to a position outside the heart and may refer to a position outside of the pericardium surrounding the heart of a patient. Extra-cardiac electrodes may be carried by a non-transvenous lead or a transvenous lead. A transvenous extra-cardiac lead may carry implantable electrodes that can be positioned intravenously but outside the heart in an extra-cardiac location, e.g., within the internal thoracic vein, jugular vein, or another vein. As used herein, the term “extra-cardiovascular” refers to a position outside the blood vessels and heart, which may also be outside the pericardium surrounding the heart of a patient. Implantable electrodes carried by non-transvenous, extra-cardiovascular leads may be positioned extra-thoracically (outside the ribcage and sternum) or intra-thoracically (beneath the ribcage or sternum) but may not be in intimate contact with myocardial tissue. In general, the techniques disclosed herein for delivering cardiac pacing pulses may be utilized in conjunction with a medical device and a low impedance pacing electrode vector that is not in contact with the myocardial tissue of the patient's heart.

As disclosed herein, a medical device includes a therapy delivery circuit including operative circuitry configured to deliver high voltage CV/DF shock pulses using high surface area, low impedance electrodes. The medical device is further configured to generate relatively lower voltage cardiac pacing pulses that are delivered via a high voltage output circuit to a high surface area, low impedance pacing electrode vector that may also be used for delivering CV/DF shock pulses. As described below, the therapy delivery circuitry of the medical device may include an internal adjustable load that may be digitally programmable for drawing a controlled current through the high voltage output circuit components that require a high operating current for being held in a conducting state for delivery of relatively low voltage cardiac pacing pulses.

The techniques disclosed herein may be implemented in any implantable, partially implantable, or external or wearable pacemaker or ICD system, e.g., in a pacemaker or ICD having extra-cardiac electrodes. The electrodes may be carried by an implantable medical electrical lead extending from the pacemaker or ICD and/or carried by the housing of the pacemaker or ICD. The techniques disclosed herein are not necessarily limited to implantable systems, however, and may be implemented in an external pacemaker or ICD using cutaneous surface electrodes or transcutaneous electrodes.

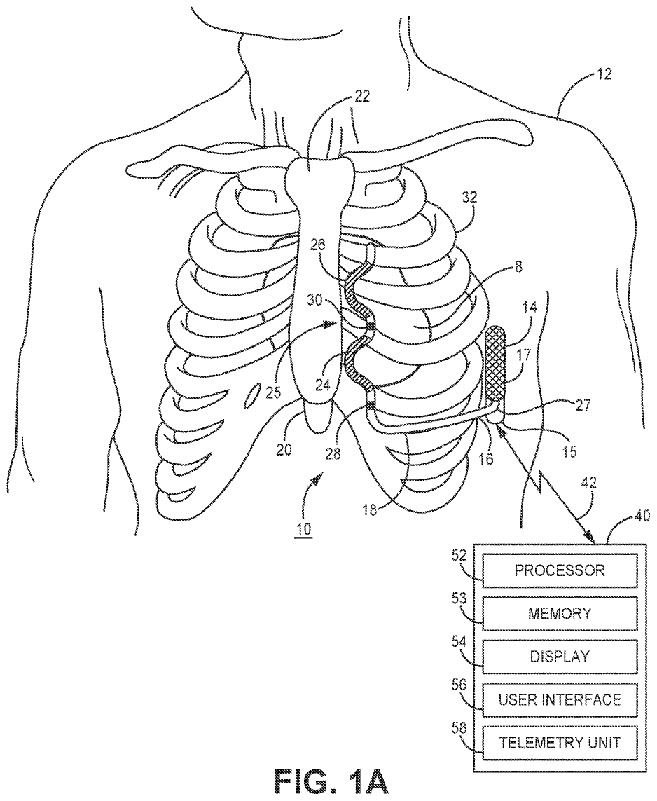

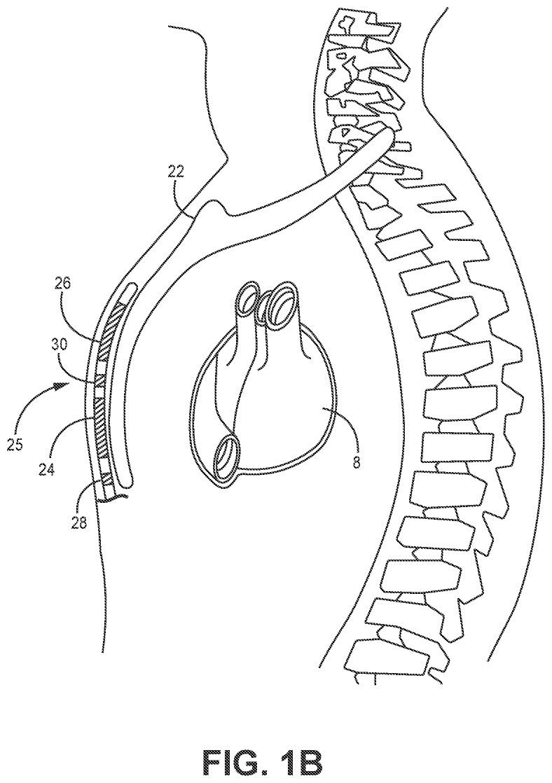

FIGS. 1A and 1B are conceptual diagrams of one example of an ICD system 10 that may be configured to sense cardiac electrical signals and deliver electrical stimulation therapy according to the techniques disclosed herein. FIG. 1A is a front view of ICD system 10 implanted within patient 12. FIG. 1B is a side view of ICD system 10 implanted within patient 12. ICD system 10 includes an ICD 14 connected to an electrical stimulation and sensing lead 16, positioned in an extra-cardiovascular location in this example. FIGS. 1A and 1B are described in the context of an ICD system 10 capable of providing high voltage CV/DF shocks and relatively lower voltage cardiac pacing pulses in response to detecting a cardiac arrhythmia based on processing of sensed cardiac electrical signals.

ICD 14 includes a housing 15 that forms a hermetic seal that protects internal components of ICD 14. The housing 15 of ICD 14 may be formed of a conductive material, such as titanium or titanium alloy. The housing 15 may function as an electrode (sometimes referred to as a “can” electrode). Housing 15 may be used as an active can electrode for use in delivering CV/DF shocks or other high voltage pulses delivered using a high voltage therapy circuit. In other examples, housing 15 may be available for use in delivering unipolar, relatively lower voltage cardiac pacing pulses and/or for sensing cardiac electrical signals in combination with electrodes carried by lead 16. In other instances, the housing 15 of ICD 14 may include a plurality of electrodes on an outer portion of the housing. The outer portion(s) of the housing 15 functioning as an electrode(s) may be coated with a material, such as titanium nitride, e.g., for reducing post-stimulation polarization artifact.

ICD 14 includes a connector assembly 17 (also referred to as a connector block or header) that includes electrical feedthroughs crossing housing 15 to provide electrical connections between conductors extending within the lead body 18 of lead 16 and electronic components included within the housing 15 of ICD 14. As will be described in further detail herein, housing 15 may house one or more processing circuits, memories, transceivers, cardiac electrical signal sensing circuitry, therapy delivery circuitry, power sources and other components for sensing cardiac electrical signals, detecting a heart rhythm, and controlling and delivering electrical stimulation pulses to treat an abnormal heart rhythm.

Elongated lead body 18 has a proximal end 27 that includes a lead connector (not shown) configured to be connected to ICD connector assembly 17 and a distal portion 25 that includes one or more electrodes. In the example illustrated in FIGS. 1A and 1B, the distal portion 25 of lead body 18 includes high surface area, low impedance electrodes 24 and 26 and relatively low surface area, higher impedance electrodes 28 and 30. Electrodes 24 and 26 are elongated electrodes that may extend along a portion of the length of lead body 18 to form a relatively high surface area, low impedance electrode that can be used for delivering high voltage CV/DF pulses. A CV shock pulse may be synchronized to an intrinsic R-wave sensed by ICD 14 for terminating non-sinus, tachycardia. A DF shock pulse may be delivered without synchronization to a sensed R-wave for terminating fibrillation. In either case, the high voltage, high energy CV/DF shock pulse is delivered to the heart using high surface area electrodes, e.g., elongated coil electrodes, to cause depolarization of a large mass of the myocardial tissue simultaneously. The simultaneous depolarization of the large mass of myocardial tissue is followed by repolarization and an associated state of physiological refractoriness of the large mass, which disrupts the conduction of aberrant depolarizations through the heart that are causing the tachyarrhythmia. In this way, the tachyarrhythmia may be successfully terminated because the heart's normal, intrinsic electrical conduction system (or a cardiac pacing pulse) may initiate the next heartbeat to restore a more normal, organized propagation and conduction of the myocardial depolarizations through the heart.

High surface area electrodes, such as electrodes 24 and 26 and/or housing 24, are used to deliver CV/DF shocks in order to encompass a large mass of the heart within the electrical field between the electrodes selected in the CV/DF electrode vector and to avoid tissue injury at the electrode sites that could occur when delivering high voltage shocks via a lower electrode surface area, resulting in a high current density at a more localized tissue site. Electrodes 24 and 26 may be configured to be activated concurrently to form one, large surface area, low impedance anode or cathode. Alternatively, electrodes 24 and 26 may form separate high surface area, low impedance electrodes in which case each of the electrodes 24 and 26 may be activated independently, e.g., as an anode or cathode, for delivering CV/DF shock pulses.

As disclosed herein, electrodes 24 and 26 may be selected in a low impedance pacing electrode vector for delivering cardiac pacing pulses, having a much lower voltage amplitude than a CV/DF shock but possibly a higher voltage than the voltage amplitude required of cardiac pacing pulses delivered using endocardial or epicardial pacing electrodes that are in intimate contact with the heart 8. One electrode 24 or 26 may serve as a pacing cathode with the other electrode 26 or 24 serving as the return anode. In other examples, one electrode 24 or 26, or concurrently selected electrodes 24 and 26, may serve as the pacing cathode with the housing 15 or another available electrode serving as the return anode electrode.

For the sake of convenience, electrodes 24 and 26 are referred to herein as “coil electrodes” because they may take the form of an elongated, coiled electrode (which may include a single wire or filar or multiple wires or filars, e.g., a braided multi-filar wire, a stranded multi-filar wire, etc.) winding around a longitudinal portion of lead body 18 to provide a relatively high surface area for delivering high voltage CV/DF shocks. However, it is to be understood that electrodes 24 and 26 may be configured as other types of high surface area electrodes that can be used for delivering CV/DF shocks, which may include ribbon electrodes, plate electrodes, serpentine electrodes, zig-zagging electrodes, or other types of physical electrode configurations that provide a relatively large surface area and low impedance and do not necessarily include a coiled wire.

Coil electrodes 24 and 26 (and in some examples housing 15) are sometimes referred to as “defibrillation electrodes” or “CV/DF electrodes” because they are utilized, individually or collectively, for delivering high voltage CV/DF shocks. However, as disclosed herein coil electrodes 24 and 26 (and in some examples housing 15) may be utilized in a cardiac pacing electrode vector to provide cardiac pacing pulse delivery. Furthermore, in some examples, coil electrodes 24 and 26 may be utilized in a sensing electrode vector for providing sensing functionality in addition to being utilized for delivering high voltage CV/DF shocks and/or cardiac pacing pulses. In this sense, the use of the term “defibrillation electrode” or “CV/DF electrode” herein should not be considered as limiting the electrodes 24 and 26 for use in only high voltage CV/DF shock therapy applications. For example, either of coil electrodes 24 and 26 may be used as a sensing electrode in a sensing electrode vector for sensing cardiac electrical signals and determining a need for an electrical stimulation therapy. Furthermore, either or both of coil electrodes 24 and 26 may be used in a cardiac pacing electrode vector for delivering cardiac pacing pulses according to the techniques disclosed herein for pacing using a low impedance pacing electrode vector. While two coil electrodes 24 and 26 are shown along lead body 18, in other examples only one coil electrode (which may be used in combination with housing 15 for delivering high voltage pulses) or three or more coil electrodes may be carried by lead body 18. In still other examples, two or more coil electrodes may be carried by two or more different lead bodies extending from ICD 14.

Electrodes 28 and 30 are relatively smaller surface area electrodes which are available for use in sensing electrode vectors for sensing cardiac electrical signals and may be used for delivering relatively low voltage cardiac pacing pulses in some examples. Electrodes 28 and 30 are sometimes referred to as “pace/sense electrodes” because they are generally configured for use in relatively low voltage applications, e.g., used as either a cathode or anode for delivery of pacing pulses and/or sensing of cardiac electrical signals, as opposed to delivering high voltage CV/DF shocks. In some instances, electrodes 28 and 30 may provide only pacing functionality, only sensing functionality or both.

Electrodes 28 and 30 may be ring electrodes extending around the circumference of lead body 18 and having a relatively short longitudinal dimension along the length of lead body 18 compared to coil electrodes 24 and 26. For the sake of convenience, electrodes 28 and 30 are referred to herein as “ring electrodes” to distinguish them from the relatively larger surface area, low impedance electrodes 24 and 26, referred to herein as “coil electrodes.” However, electrodes 28 and 30 may comprise any of a number of different types of electrodes, including ring electrodes, short coil electrodes, button electrodes, hemispherical electrodes, directional electrodes, segmented electrodes, helical electrodes, fishhook electrodes, tip electrodes, or the like and are not limited to being exclusively ring electrodes.

In the example illustrated in FIGS. 1A and 1B, ring electrode 28 is located proximal to coil electrode 24, and ring electrode 30 is located between coil electrodes 24 and 26. Ring electrodes 28 and 30 may be positioned at other locations along lead body 18 and are not limited to the positions shown. One, two or more ring or other low surface area electrodes used for sensing and/or low voltage cardiac pacing pulse delivery may be carried by lead body 18. For instance, a third ring electrode may be located distal to coil electrode 26 in some examples. In other examples, lead 16 may include fewer or more ring electrodes and/or coil electrodes than the example shown here.

In some cases, post-shock cardiac pacing pulses are needed to prevent asystole following a CV/DF shock until the intrinsic conduction system initiates an intrinsic heart rhythm. In other cases, cardiac pacing may be needed to treat bradycardia, asystole or deliver anti-tachycardia pacing (ATP), as examples. Cardiac pacing pulses are generally much lower in voltage than CV/DF shock pulses because a much smaller, relatively local volume of cardiac tissue can be captured by a pacing pulse to cause a heartbeat than the relatively large mass of cardiac tissue that is simultaneously depolarized during a CV/DF shock. Cardiac pacing pulses are delivered to cause depolarization of myocardial tissue at one or more local pacing sites. The pacing evoked depolarization of local cardiac cells captured in the vicinity of the current field of the pacing cathode electrode is conducted through the heart via the myocardium in a coordinated manner to cause a paced heartbeat.

In some pacemaker and ICD systems, cardiac pacing pulses can be delivered using relatively low surface area electrodes, similar to that of ring electrodes 28 and 30, carried by endocardial or epicardial leads so that the low surface area electrodes are in close or intimate contact with myocardial tissue. Pacing pulses delivered using low surface area, transvenous, endocardial electrodes, for example, may typically have a voltage amplitude of 8 volts (V) or less and a pulse width of 2.0 ms or less. More typically, a pacing pulse that successfully paces the heart via endocardial or epicardial electrodes might be 1.0 to 5.0 V, e.g., 2.5 V, in pulse amplitude with a 0.25 to 0.5 ms pulse width, as illustrative examples. The pulse amplitude and pulse width of the pacing pulse are selected to deliver sufficient energy to cause electrical depolarization of the myocardial tissue of the heart at the pacing site to thereby capture the heart and cause a heartbeat.

Cardiac pacing pulses that are delivered using extra-cardiac electrodes that are not in contact with cardiac tissue generally require higher energy (e.g., higher pulse amplitude and/or pulse width) than cardiac pacing pulses that are delivered using endocardial or epicardial electrodes. However, these cardiac pacing pulses delivered using extra-cardiac electrodes are still much lower in voltage amplitude and overall pulse energy than that required for CV/DF shocks. Relatively higher voltage cardiac pacing pulses are required when pacing using extracardiac electrodes than endocardial or epicardial electrodes in order to deliver enough energy within the pacing pulse width to capture the heart. A limitation of the maximum pacing pulse width may exist due in part to the decay rate of the pacing pulse amplitude delivered by the ICD therapy delivery circuitry. The decay rate can be dependent on the capacitance of a holding capacitor being discharged to deliver the pacing pulse and the impedance of the pacing electrode vector. In order to achieve capture within a limited pulse width, e.g., 8 ms or less, 4 ms or less or 2 ms or less, a high pacing voltage amplitude may be required to deliver sufficient pacing pulse energy. Cardiac pacing pulses delivered using extra-cardiac electrodes may be in the range of 8 V to 40 V with a pacing pulse width of 2 ms to 8 ms, as examples. By comparison CV/DF shocks may be greater than 100 V or on the order of several hundred volts.

As described below, high surface area coil electrodes 24 and 26 may be employed for delivering cardiac pacing pulses. Relatively higher pacing pulse voltage amplitudes may be used with lower current density at the electrode tissue interface of the high surface area coil electrodes 24 and 26 compared to the low surface area electrodes 28 and 30. The surface area of a coil electrode 24 or 26 may be 50 to 100 times larger than the surface area of the ring electrodes 28 and 30. High current density at the ring electrode-tissue interface during relatively high voltage cardiac pacing could cause local tissue injury. The electrical field of current traveling through conductive tissues toward the heart between a cardiac pacing electrode vector that includes at least one or both high surface area coil electrodes 24 and 26 may be more effective in capturing the heart for cardiac pacing than the electrical field between a cardiac pacing electrode vector that includes lower surface area ring electrodes 28 and 30 or one of ring electrodes 28 or 30 and housing 15. A higher voltage cardiac pacing pulse that can be delivered via the coil electrodes 24 and/or 26 and/or housing 15 can have a relatively short pulse width so that the pacing pulse decay rate does not become a limiting factor of pacing pulse energy delivered for capturing the heart.

Accordingly, as described below, ICD 14 may be configured to deliver cardiac pacing pulses using coil electrodes 24 and/or 26, e.g., as a cathode and anode pair. High voltage output circuitry of ICD 14 is enabled by therapy delivery control circuitry of ICD 14 when a CV/DF shock is needed for delivery via coil electrodes 24 and/or 26. However, when a cardiac pacing pulse is needed, that is a much lower voltage than the CV/DF shock pulse, ICD 14 may be configured to enable the high voltage output circuitry for delivering a cardiac pacing pulse using a low impedance pacing electrode vector that includes one or both of coil electrodes 24 and 26. Current required to operate the high voltage output circuitry is controlled using an internal adjustable load that is electrically connected in parallel with electrode terminals that can be coupled to the external pacing load. The internal adjustable load is configured to pull a controlled, adjustable current that maintains charge coupled components of the high voltage output circuitry in a conducting state for delivery of cardiac pacing pulses.

Referring again to the example shown in FIGS. 1A and 1B, lead 16 extends subcutaneously or submuscularly over the ribcage 32 medially from the connector assembly 27 of ICD 14 toward a center of the torso of patient 12, e.g., toward xiphoid process 20 of patient 12. At a location near xiphoid process 20, lead 16 bends or turns and extends superiorly, subcutaneously or submuscularly, over the ribcage and/or sternum, substantially parallel to sternum 22. Although illustrated in FIG. 1A as being offset laterally from and extending substantially parallel to sternum 22, the distal portion 25 of lead 16 may be implanted at other locations, such as over sternum 22, offset to the right or left of sternum 22, angled laterally from sternum 22 toward the left or the right, or the like. Alternatively, lead 16 may be placed along other subcutaneous or submuscular paths. The path of extra-cardiovascular lead 16 may depend on the location of ICD 14, the arrangement and position of electrodes carried by the lead body 18, and/or other factors. The techniques disclosed herein are not limited to a particular path of lead 16 or final locations of electrodes 24, 26, 28 and 30.

Electrical conductors (not illustrated) extend through one or more lumens of the elongated lead body 18 of lead 16 from the lead connector at the proximal lead end 27 to electrodes 24, 26, 28, and 30 located along the distal portion 25 of the lead body 18. The elongated electrical conductors contained within the lead body 18, which may be separate respective insulated conductors within the lead body 18, are each electrically coupled with respective coil electrodes 24 and 26 and ring electrodes 28 and 30. The respective conductors electrically couple the electrodes 24, 26, 28, and 30 to circuitry, such as a therapy delivery circuit and/or a sensing circuit, of ICD 14 via connections in the connector assembly 17, including associated electrical feedthroughs crossing housing 15. The electrical conductors transmit electrical stimulation pulses from therapy delivery circuitry within ICD 14 to one or more of coil electrodes 24 and 26 and/or ring electrodes 28 and 30 and transmit electrical signals produced by the patient's heart 8 from one or more of coil electrodes 24 and 26 and/or ring electrodes 28 and 30 to the sensing circuitry within ICD 14.

The lead body 18 of lead 16 may be formed from a non-conductive material, including silicone, polyurethane, fluoropolymers, mixtures thereof, and/or other appropriate materials, and shaped to form one or more lumens within which the one or more conductors extend. Lead body 18 may be tubular or cylindrical in shape. In other examples, the distal portion 25 (or all of) the elongated lead body 18 may have a flat, ribbon or paddle shape. Lead body 18 may be formed having a preformed distal portion 25 that is generally straight, curving, bending, serpentine, undulating or zig-zagging.

In the example shown, lead body 18 includes a curving distal portion 25 having two “C” shaped curves, which together may resemble the Greek letter epsilon, “ε.” Defibrillation electrodes 24 and 26 are each carried by one of the two respective C-shaped portions of the lead body distal portion 25. The two C-shaped curves are seen to extend or curve in the same direction away from a central axis of lead body 18, along which ring electrodes 28 and 30 are positioned. Ring electrodes 28 and 30 may, in some instances, be approximately aligned with the central axis of the straight, proximal portion of lead body 18 such that mid-points of coil electrodes 24 and 26 are laterally offset from ring electrodes 28 and 30.

Other extra-cardiovascular leads including one or more coil or other high surface area electrodes and optionally one or more ring or other relatively low surface area may be implemented with the techniques described herein. The techniques disclosed herein are not limited to any particular lead body design. In other examples, lead body 18 can be a flexible elongated lead body without any pre-formed shape, bends or curves.

ICD 14 may obtain cardiac electrical signals corresponding to electrical activity of heart 8 via a combination of sensing electrode vectors that include combinations of electrodes 24, 26, 28 and/or 30. In some examples, housing 15 of ICD 14 is used in combination with one or more of electrodes 24, 26, 28 and/or 30 in at least one sensing electrode vector. Each cardiac electrical signal received via a selected sensing electrode vector may be used by ICD 14 for sensing cardiac event signals attendant to intrinsic depolarizations of the myocardium, e.g., R-waves attendant to ventricular depolarizations and in some cases P-waves attendant to atrial depolarizations. Sensed cardiac event signals may be used for determining the heart rate and determining a need for cardiac pacing, e.g., for treating bradycardia or asystole for preventing a long ventricular pause, or for determining a need for tachyarrhythmia therapy, e.g., ATP and/or CV/DF shocks.

ICD 14 analyzes the cardiac electrical signal(s) received from one or more sensing electrode vectors to monitor for abnormal rhythms, such as asystole, bradycardia, ventricular tachycardia (VT) and/or ventricular fibrillation (VF). ICD 14 may analyze the heart rate and/or morphology of the cardiac electrical signals to monitor for tachyarrhythmia in accordance with any tachyarrhythmia detection techniques. ICD 14 generates and delivers electrical stimulation therapy in response to detecting a tachyarrhythmia, e.g., VT or VF (VT/VF) using a therapy delivery electrode vector which may be selected from any of the available electrodes 24, 26, 28 30 and/or housing 15. ICD 14 may deliver ATP in response to VT detection and in some cases may deliver ATP prior to a CV/DF shock or during high voltage holding capacitor charging in an attempt to avert the need for delivering a CV/DF shock. If ATP does not successfully terminate VT or when VF is detected, ICD 14 may deliver one or more CV/DF shocks via one or both of coil electrodes 24 and 26 and/or housing 15.

In the absence of a ventricular event signal, e.g., a sensed R-wave, ICD 14 may generate and deliver a cardiac pacing pulse, such as a post-shock pacing pulse or bradycardia pacing pulse. When asystole is detected or when a pacing escape interval expires prior to sensing a ventricular event signal (e.g., and R-wave), one or more cardiac pacing pulses may be delivered by ICD 14. The cardiac pacing pulses may be delivered using a low impedance pacing electrode vector that includes at least one or both coil electrodes 24 and 26 according to the techniques disclosed herein. In some examples, housing 15 of ICD 14 is used in combination with one or both coil electrodes 24 and 26 to deliver cardiac pacing pulses.

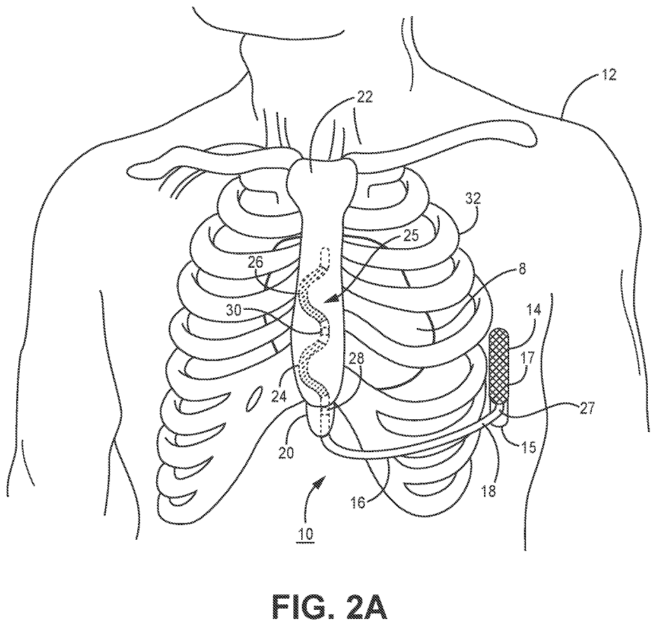

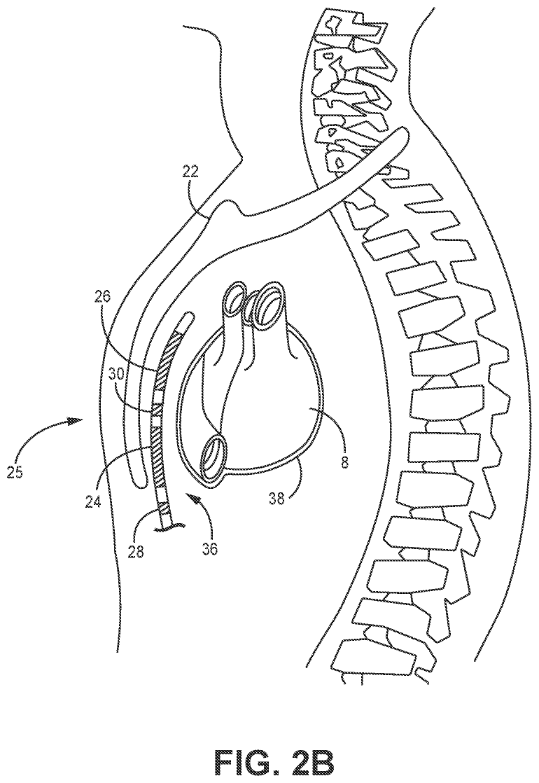

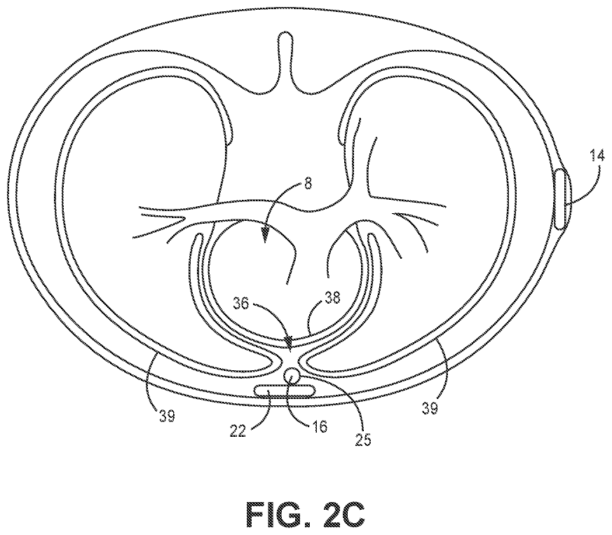

ICD 14 is shown implanted subcutaneously on the left side of patient 12 along the ribcage 32. ICD 14 may, in some instances, be implanted between the left posterior axillary line and the left anterior axillary line of patient 12. ICD 14 may, however, be implanted at other subcutaneous or submuscular locations in patient 12. For example, ICD 14 may be implanted in a subcutaneous pocket in the pectoral region. In this case, lead 16 may extend subcutaneously or submuscularly from ICD 14 toward the manubrium of sternum 22 and bend or turn and extend inferiorly from the manubrium to the desired location subcutaneously or submuscularly. In yet another example, ICD 14 may be placed abdominally. Lead 16 may be implanted in other extra-cardiovascular locations as well. For instance, as described with respect to FIGS. 2A-2C, the distal portion 25 of lead 16 may be implanted underneath the sternum/ribcage in the substernal space. FIGS. 1A and 1B are illustrative in nature and should not be considered limiting in the practice of the techniques disclosed herein.

A medical device operating according to techniques disclosed herein may be coupled to one or more transvenous or non-transvenous leads in various examples for carrying electrodes for sensing cardiac electrical signals and delivering electrical stimulation therapy. For example, the medical device, such as ICD 14, may be coupled to an extra-cardiovascular lead as illustrated in the accompanying drawings, referring to a lead that positions electrodes outside the blood vessels, heart, and pericardium surrounding the heart of a patient. Implantable electrodes carried by extra-cardiovascular leads may be positioned extra-thoracically (outside the ribcage and sternum), subcutaneously or submuscularly, or intra-thoracically (beneath the ribcage or sternum, sometimes referred to as a sub-sternal position) and may not necessarily be in intimate contact with myocardial tissue. An extra-cardiovascular lead may also be referred to as a “non-transvenous” lead.

In other examples, the medical device may be coupled to a transvenous lead that positions electrodes within a blood vessel, which may remain outside the heart in an extra-cardiac location. For instance, a transvenous medical lead may be advanced along a venous pathway to position electrodes in an extra-cardiac location within the internal thoracic vein (ITV), an intercostal vein, the superior epigastric vein, or the azygos, hemiazygos, or accessory hemiazygos veins, as examples. In still other examples, a transvenous lead may be advanced to position electrodes within the heart, e.g., within an atrial and/or ventricular heart chamber.

An external device 40 is shown in telemetric communication with ICD 14 by a wireless communication link 42 in FIG. 1A. External device 40 may include a processor 52, memory 53, display unit 54, user interface 56 and telemetry unit 58. Processor 52 controls external device operations and processes data and signals received from ICD 14. Display unit 54, which may include a graphical user interface, displays data and other information to a user for reviewing ICD operation and programmed parameters as well as cardiac electrical signals retrieved from ICD 14.

User interface 56 may include a mouse, touch screen, keypad or the like to enable a user to interact with external device 40 to initiate a telemetry session with ICD 14 for retrieving data from and/or transmitting data to ICD 14, including programmable parameters for controlling cardiac event signal sensing, determining a need for electrical stimulation therapy, and for therapy delivery. Telemetry unit 58 includes a transceiver and antenna configured for bidirectional communication with a telemetry circuit included in ICD 14 and is configured to operate in conjunction with processor 52 for sending and receiving data relating to ICD functions via communication link 42.

Communication link 42 may be established between ICD 14 and external device 40 using a radio frequency (RF) link such as BLUETOOTH®, Wi-Fi, or Medical Implant Communication Service (MICS) or other RF or communication frequency bandwidth or communication protocols. Data stored or acquired by ICD 14, including physiological signals or associated data derived therefrom, results of device diagnostics, battery status, and histories of detected rhythm episodes and delivered therapies, etc., may be retrieved from ICD 14 by external device 40 following an interrogation command.

External device 40 may be embodied as a programmer used in a hospital, clinic or physician's office to retrieve data from ICD 14 and to program operating parameters and algorithms in ICD 14 for controlling ICD functions. External device 40 may alternatively be embodied as a home monitor or handheld device. At least some control parameters used in sensing cardiac event signals and detecting arrhythmias as well as therapy delivery control parameters may be programmed into ICD 14 using external device 40 in some examples. For example, a user may program a pacing voltage amplitude and pacing electrode vector that includes at least one or both coil electrodes 24 and 26. As described below, processing and control circuitry enclosed by housing 15 may select a cardiac pacing pulse voltage source and control therapy output circuitry for delivering cardiac pacing pulses via at least one coil electrode 24 or 26 based on the programmed pacing pulse voltage amplitude.

FIGS. 2A-2C are conceptual diagrams of patient 12 implanted with extra-cardiovascular ICD system 10 in a different implant configuration than the arrangement shown in FIGS. 1A-1B. FIG. 2A is a front view of patient 12 implanted with ICD system 10. FIG. 2B is a side view of patient 12 implanted with ICD system 10. FIG. 2C is a transverse view of patient 12 implanted with ICD system 10. In this arrangement, lead 16 of system 10 is implanted at least partially underneath sternum 22 of patient 12. Lead 16 extends subcutaneously or submuscularly from ICD 14 toward xiphoid process 20 and at a location near xiphoid process 20 bends or turns and extends superiorly within anterior mediastinum 36 (see FIG. 2C) in a substernal position.

Anterior mediastinum 36 may be viewed as being bounded laterally by pleurae 39, posteriorly by pericardium 38, and anteriorly by sternum 22 (see FIG. 2C). The distal portion 25 of lead 16 may extend along the posterior side of sternum 22 substantially within the loose connective tissue and/or substernal musculature of anterior mediastinum 36. A lead implanted such that the distal portion 25 is substantially within anterior mediastinum 36, may be referred to as a “substernal lead.”

In the example illustrated in FIGS. 2A-2C, lead 16 is located substantially centered under sternum 22. In other instances, however, lead 16 may be implanted such that it is offset laterally from the center of sternum 22. In some instances, lead 16 may extend laterally such that distal portion 25 of lead 16 is underneath/below the ribcage 32 in addition to or instead of sternum 22. In other examples, the distal portion 25 of lead 16 may be implanted in other extra-cardiac, intra-thoracic locations, including in the pleural cavity or around the perimeter of and adjacent to the pericardium 38 of heart 8.

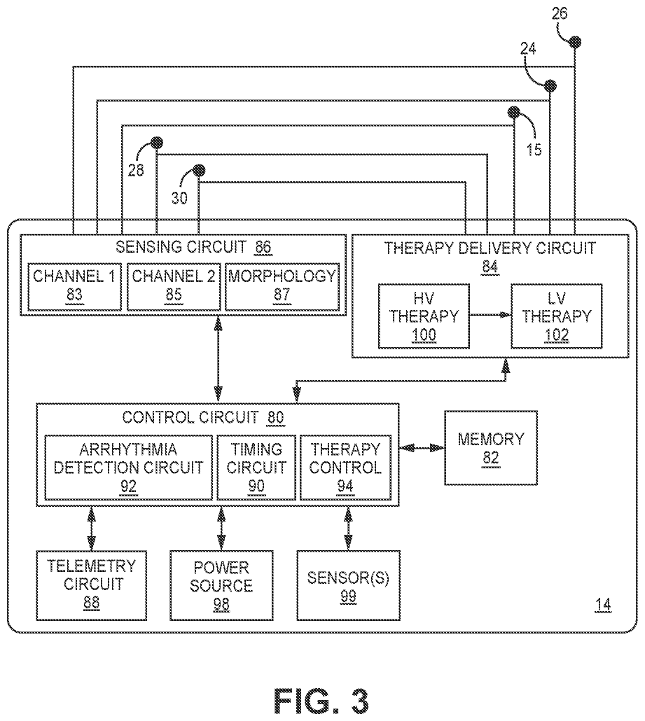

FIG. 3 is a conceptual diagram of ICD 14 according to one example. The electronic circuitry enclosed within housing 15 (shown schematically in FIG. 3 as an electrode, sometimes referred to as a “can electrode”) includes software, firmware and hardware that cooperatively monitor cardiac electrical signals, determine when an electrical stimulation therapy is necessary, and deliver therapy as needed according to programmed therapy delivery algorithms and control parameters. ICD 14 may be coupled to a lead, such as lead 16 carrying electrodes 24, 26, 28, and 30 as shown in the examples of FIGS. 1A-2C, for delivering electrical stimulation pulses to the patient's heart and for sensing cardiac electrical signals.

ICD 14 may include a control circuit 80, memory 82, therapy delivery circuit 84, cardiac electrical signal sensing circuit 86, telemetry circuit 88, and, in some examples, one or more physiological sensors 99. A power source 98 provides power to the circuitry of ICD 14, including each of the components 80, 82, 84, 86, and 88 as needed. Power source 98 may include one or more energy storage devices, such as one or more rechargeable or non-rechargeable batteries. The connections between power source 98 and each of the other components 80, 82, 84, 86 and 88 are to be understood from the general block diagram of FIG. 3 but are not shown for the sake of clarity. For example, power source 98 may be coupled to one or more charging circuits included in therapy delivery circuit 84 for charging holding capacitors included in therapy delivery circuit 84 and operating output circuitry for discharging the holding capacitor(s) at appropriate times under the control of control circuit 80 for producing electrical pulses according to a therapy protocol. Power source 98 is also coupled to components of cardiac electrical signal sensing circuit 86 (such as sense amplifiers, analog-to-digital converters, switching circuitry, etc.), memory 82, and telemetry circuit 88 as needed.

The various operating circuits shown in FIG. 3 represent functionality included in ICD 14 and may include any discrete and/or integrated electronic circuit components that implement analog and/or digital circuits capable of producing the functions attributed to ICD 14 herein. Functionality associated with one or more circuits may be performed by separate hardware, firmware and/or software components, or integrated within common hardware, firmware and/or software components. For example, cardiac electrical signal sensing and analysis for detecting arrhythmia may be performed cooperatively by sensing circuit 86 and control circuit 80 and may include operations implemented in a processor or other signal processing circuitry included in control circuit 80 executing instructions stored in memory 82 and control signals such as blanking and timing intervals and sensing threshold amplitude signals sent from control circuit 80 to sensing circuit 86. Therapy delivery may be performed cooperatively by therapy delivery circuit 84 under the control of signals received from control circuit 80 for controlling the timing, amplitude, width, polarity, rate, electrode vector and other therapy delivery parameters used by therapy delivery circuit 84 to generate and deliver electrical stimulation pulses, which may include CV/DF pulses, cardiac pacing pulses, tachyarrhythmia induction pulses, impedance measurement pulses or any other electrical pulses delivered via electrodes 24, 26, 28, 30 and/or housing 15.

The various circuits of ICD 14 may include an application specific integrated circuit (ASIC), an electronic circuit, a processor (shared, dedicated, or group) and memory that execute one or more software or firmware programs, a combinational logic circuit, state machine, hardware subroutine, or other suitable components or combinations of components that provide the described functionality. The particular form of software, hardware and/or firmware employed to implement the functionality disclosed herein will be determined primarily by the particular system architecture employed in the ICD and by the particular sensing, detection and therapy delivery methodologies employed by the ICD. Providing software, hardware, and/or firmware to accomplish the described functionality in the context of any modern medical device system, given the disclosure herein, is within the abilities of one of skill in the art.

Memory 82 may include any volatile, non-volatile, magnetic, or electrical non-transitory computer readable storage media, such as random access memory (RAM), read-only memory (ROM), non-volatile RAM (NVRAM), electrically-erasable programmable ROM (EEPROM), flash memory, or any other memory device. Furthermore, memory 82 may include non-transitory computer readable media storing instructions that, when executed by one or more processing circuits, cause control circuit 80 and/or other ICD components to perform various functions attributed to ICD 14 or those ICD components. The non-transitory computer-readable media storing the instructions may include any of the media listed above.

Therapy delivery circuit 84 and sensing circuit 86 are electrically coupled to electrodes 24, 26, 28, 30 carried by lead 16 and the housing 15, which may function as a common or ground electrode for sensing or cardiac pacing pulses or as an active can electrode for delivering CV/DF shock pulses or cardiac pacing pulses. Control circuit 80 communicates, e.g., via a data bus, with therapy delivery circuit 84 and sensing circuit 86 for sensing cardiac electrical signals, detecting cardiac rhythms, and controlling delivery of cardiac electrical stimulation therapies in response to sensed cardiac signals (or the absence thereof). Control circuit 80 may include an arrhythmia detection circuit 92, timing circuit 90, and therapy control circuit 94. Arrhythmia detection circuit 92 may be configured to process and analyze signals received from sensing circuit 86, which may be in conjunction with time intervals and/or timing related signals received from timing circuit 90. Timing circuit 90 may generate clock signals and include various timers and/or counters for use in determining time intervals between cardiac events, sensed and/or paced, and control the timing of delivered pacing pulses and/or CV shocks. Control circuit 80 may further include a therapy control circuit 94 configured to pass signals to and receive signals from therapy delivery circuit 84 for controlling and monitoring electrical stimulation therapies delivered by therapy delivery circuit 84.

Cardiac electrical signal sensing circuit 86 (also referred to herein as “sensing circuit” 86) may be selectively coupled to electrodes 28, 30 and/or housing 15 in order to monitor electrical activity of the patient's heart. Sensing circuit 86 may additionally be selectively coupled to coil electrodes 24 and/or 26 for use in a sensing electrode vector together or in combination with one or more of electrodes 28, 30 and/or housing 15. Sensing circuit 86 may be enabled to receive cardiac electrical signals from at least one sensing electrode vector selected from the available electrodes 24, 26, 28, 30, and housing 15 in some examples. At least two, three or more cardiac electrical signals from two, three or more different sensing electrode vectors may be received simultaneously by sensing circuit 86 in some examples. Sensing circuit 86 may monitor one or more cardiac electrical signals for sensing cardiac event signals, e.g., R-waves attendant to intrinsic ventricular myocardial depolarizations. In some examples, sensing circuit 86 may be configured to monitor two cardiac electrical signals simultaneously for sensing cardiac event signals. At least one cardiac electrical signal may be received by sensing circuit 86 and passed to control circuit 80 for processing and analysis, e.g., by arrhythmia detection circuit 92, for determining when morphology-based criteria for detecting arrhythmia are met in some examples.

In the example shown, sensing circuit 86 may include switching circuitry for selecting which of electrodes 24, 26, 28, 30, and housing 15 are coupled as a first sensing electrode vector to a first sensing channel 83 for receiving a first cardiac electrical signal, which electrodes are coupled as a second sensing electrode vector to a second sensing channel 85 of sensing circuit 86 for receiving a second cardiac electrical signal, and which electrodes are coupled as a third sensing electrode vector to a morphology signal channel 87 for receiving a third cardiac electrical signal.

Each sensing channel 83 and 85, when included, may be configured to amplify, filter and digitize the cardiac electrical signal received from selected electrodes coupled to the respective sensing channel to improve the signal quality for sensing cardiac event signals, such as R-waves. The cardiac event detection circuitry within sensing circuit 86 may include one or more sense amplifiers, filters, rectifiers, threshold detectors, comparators, analog-to-digital converters (ADCs), timers or other analog and/or digital components. A cardiac event sensing threshold may be automatically adjusted by each sensing channel 83 and 85 under the control of control circuit 80, based on sensing threshold control parameters, such as various timing intervals and sensing threshold amplitude values that may be determined by control circuit 80, stored in memory 82, and/or controlled by hardware, firmware and/or software of control circuit 80 and/or sensing circuit 86. In response to sensing a cardiac event signal, e.g., an R-wave, sensing circuit 86 may generate a sensed event signal, e.g., a ventricular sensed event signal, that is passed to control circuit 80.

Ventricular sensed event signals received from sensing circuit 86 by control circuit 80 can be used by control circuit 80 for determining sensed event intervals, which can be referred to as RR intervals (RRIs). An RRI is the time interval between two ventricular sensed event signals received by control circuit 80. Control circuit 80 may include a timing circuit 90 for determining RRIs. Based on RRIs, control circuit 80 may detect VT/VF in some examples. RRIs may include time intervals between consecutive ventricular sensed event signals and intervals between a delivered pacing pulse and a ventricular sensed event signal.

In some examples, sensing circuit 86 receives a third cardiac electrical signal by morphology signal channel 87 for passing a digitized electrocardiogram (ECG) signal to control circuit 80 for morphology analysis. The three cardiac electrical signals sensed by sensing circuit 86 may be received using three different sensing electrode vectors selected from the available electrodes 24, 26, 28 and 30 and housing 15. In other examples, two cardiac electrical signals may be received by sensing circuit 86 from two different sensing electrode vectors, with one signal passed to the first sensing channel 83 and the other signal passed to the second sensing channel 85. Either or both of the two signals may be passed to control circuit 80 as a multi-bit digital ECG signal used by control circuit 80 for morphology analysis of the cardiac signal. Multiple channels 83, 85 and 87 may be optional in some examples, however. Aspects of the techniques disclosed herein for delivering therapeutic electrical stimulation pulses may be implemented in conjunction with a variety of cardiac event signal sensing and arrhythmia detection methods and are not limited to any particular method for determining the need or timing of a cardiac electrical stimulation pulse delivered by therapy delivery circuit 84.

Timing circuit 90 may be configured to control various timers and/or counters used in setting various intervals and windows used in sensing ventricular event signals, determining time intervals between received ventricular sensed event signals, performing morphology analysis and controlling the timing of cardiac pacing pulses and other electrical pulses generated by therapy delivery circuit 84. Timing circuit 90 may start a timer in response to receiving ventricular sensed event signals from sensing channels 83 and 85 and for timing RRIs. Timing circuit 90 may pass the RRIs to arrhythmia detection circuit 92 for determining and counting tachyarrhythmia intervals.

Control circuit 80 may include an arrhythmia detection circuit 92 configured to analyze RRIs received from timing circuit 90 and cardiac electrical signals received from morphology signal channel 87 for detecting arrhythmia. Arrhythmia detection circuit 92 may be configured to detect asystole and/or tachyarrhythmia based on sensed cardiac electrical signals meeting respective asystole or tachyarrhythmia detection criteria. Arrhythmia detection circuit 92 may be implemented in control circuit 80 as hardware, software and/or firmware that processes and analyzes signals received from sensing circuit 86 for detecting VT/VF. In some examples, arrhythmia detection circuit 92 may include comparators and counters for counting RRIs determined by timing circuit 90 that are tachyarrhythmia intervals. An RRI that is less than the tachyarrhythmia detection interval is referred to as a “tachyarrhythmia interval.” Arrhythmia detection circuit 92 may compare the RRIs determined by timing circuit 90 to one or more tachyarrhythmia detection interval zones, such as a VT detection interval zone and a VF detection interval zone. RRIs falling into a detection interval zone are counted by a respective VT interval counter or VF interval counter and in some cases in a combined VT/VF interval counter. When a threshold number of tachyarrhythmia intervals is reached, control circuit 80 may detect VT or VF. In some examples, a tachyarrhythmia detection based on the threshold number of tachyarrhythmia intervals being reached may be confirmed or rejected based on morphology analysis of a cardiac electrical signal.

The VF detection interval threshold may be set to 280 to 350 milliseconds (ms), as examples. When VT detection is enabled, the VT detection interval may be programmed to be in the range of 350 to 420 ms, or 400 ms as an example. VT or VF may be detected when the respective VT or VF interval counter (or a combined VT/VF interval counter) reaches a threshold number of intervals to detect (NID). As an example, the NID to detect VT may require that the VT interval counter reaches 18, 24, 32 or other selected number of VT intervals. The VT intervals may or may not be required to be consecutive intervals. The NID required to detect VF may be programmed to a threshold number of X VF intervals out of Y consecutive RRIs. For instance, the NID required to detect VF may be 18 VF intervals out of the most recent 24 consecutive RRIs, 30 VF intervals out 40 consecutive RRIs, or as high as 120 VF intervals out of 160 consecutive RRIs as examples.

Arrhythmia detection circuit 92 may be configured to perform other signal analysis for determining if other detection criteria are satisfied before detecting VT or VF based on an NID being reached, such as R-wave morphology criteria, onset criteria, stability criteria and noise and oversensing rejection criteria. To support these additional analyses, sensing circuit 86 may pass a digitized ECG signal to control circuit 80, e.g., from morphology signal channel 87, for morphology analysis performed by arrhythmia detection circuit 92 for detecting and discriminating heart rhythms. A cardiac electrical signal received by the morphology signal channel 87 (and/or sensing channel 83 and/or sensing channel 85) may be passed through a filter and amplifier, provided to a multiplexer and thereafter converted to a multi-bit digital signal by an analog-to-digital converter, all included in sensing circuit 86, for storage in memory 82. Memory 82 may include one or more circulating buffers to temporarily store digital cardiac signal segments for analysis performed by control circuit 80. Control circuit 80 may be a microprocessor-based controller that employs digital signal analysis techniques to characterize the digitized signals stored in memory 82 to recognize and classify the patient's heart rhythm employing any of numerous signal processing methodologies for analyzing cardiac signals and cardiac event waveforms, e.g., R-waves.

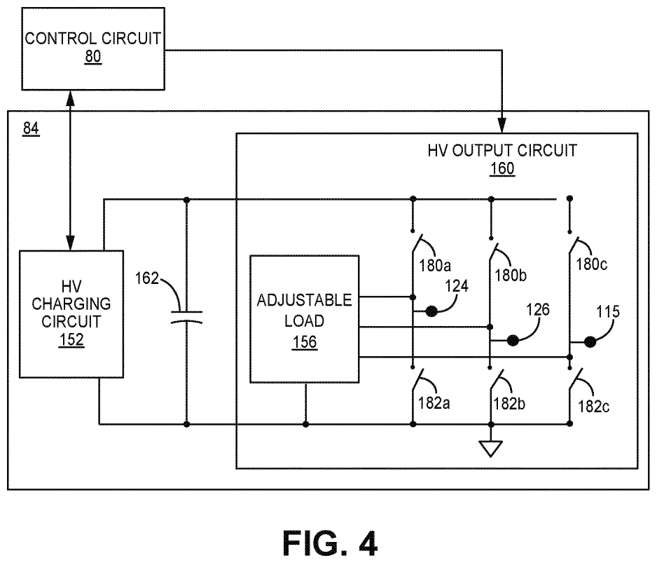

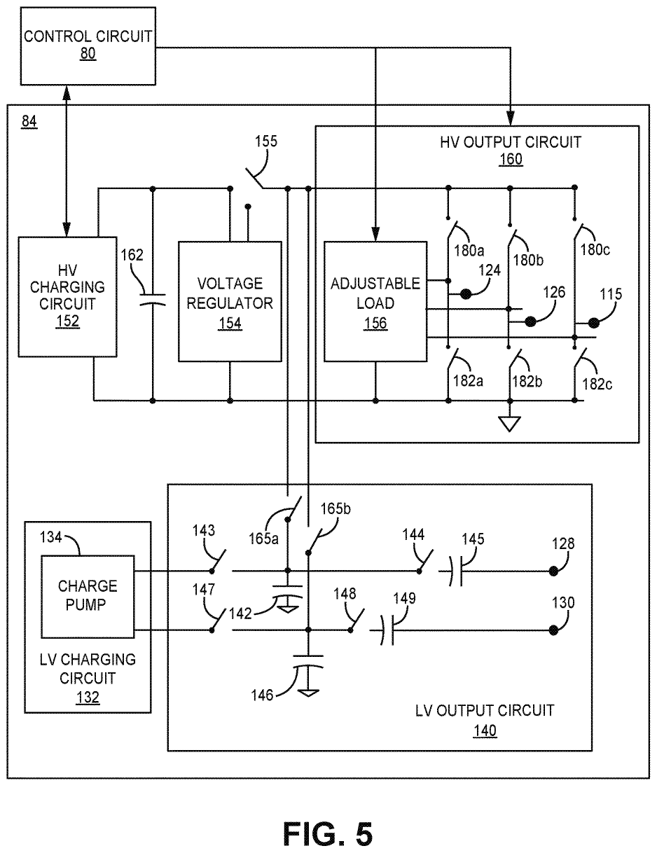

Therapy delivery circuit 84 may include at least one charging circuit and one or more charge storage devices such as one or more high voltage capacitors for generating high voltage shock pulses for treating VT/VF. Therapy delivery circuit 84 may include a high voltage (HV) therapy circuit 100, which may include a HV charging circuit, HV holding capacitor(s), and HV output circuit that are operatively controlled by signals from control circuit 80 for charging and subsequently discharging the high voltage capacitor(s) for CV/DF shock delivery when control circuit 80 detects VT/VF. Examples of circuitry that may be included in therapy delivery circuit 84 are described below in conjunction with FIGS. 4 and 5.

In some examples, therapy delivery circuit 84 may include a low voltage (LV) therapy delivery circuit 102, which may include a LV charging circuit, one or more LV holding capacitors and a LV output circuit for generating and delivering low voltage cardiac pacing pulses, e.g., cardiac pacing pulses having a pacing pulse amplitude that is 8 V or less, up to 10 V, up to 12 V, up to 16 V, or other maximum voltage amplitude of the LV therapy delivery circuit 102. LV cardiac pacing pulses may be delivered via ring electrodes 28 and/or 30 (together or in combination with housing 15) in some instances for successfully capturing and pacing the heart. Composite cardiac pacing pulses may be delivered by LV therapy delivery circuit 102 in some examples for delivering successive low voltage cardiac pacing pulses having a relatively long cumulative pulse width, e.g., up to 4 to 8 ms as examples, for delivering sufficient pulse energy to capture and pace the heart. Methods and devices for delivering composite cardiac pacing pulses, sometimes referred to as “stacked pacing pulses,” are generally disclosed in U.S. Pat. No. 10,449,362 (Anderson, et al.), incorporated herein by reference in its entirety.

In some patients, the cardiac pacing capture threshold may require a pacing pulse amplitude and/or pulse width that is greater than a maximum pacing pulse amplitude and/or pulse width that can be generated and delivered by the LV therapy delivery circuit 102 via ring electrodes 28 and 30 for successfully capturing the heart. The pacing capture threshold and/or other factors, such as the electrical field of the pacing electrode vector relative to the patient's heart, current density at the electrode tissue interface, or extraneous capture of non-cardiac tissue may make cardiac pacing via a low impedance pacing electrode vector including coil electrode 24 and/or coil electrode 26 desirable or preferred.

Cardiac pacing pulses using the high surface area coil electrodes 24 and 26 that are used to deliver CV/DF shock pulses may successfully capture the heart without limitations that may be associated with delivering cardiac pacing pulses from the LV therapy circuit 102 via the relatively small surface area ring electrodes 28 and 30 implanted at an extra-cardiac location. Delivery of cardiac pacing pulses by the HV therapy circuit 100, however, may prematurely drain current from power source 98. As further described below in conjunction with FIG. 4, HV output circuitry included in HV therapy circuit 100 may include switches and/or other charge coupled components that require a relatively high operating current for enabling delivery of a CV/DF shock. CV/DF shocks are generally delivered relatively infrequently such that the current required to operate HV output circuitry may be acceptable over the usable life of ICD 14. The voltage of cardiac pacing pulses, even when delivered at relatively high voltage amplitudes for cardiac pacing such as up to 20 Volts, 30 Volts or 40 Volts, may result in insufficient current flow through the HV output circuitry to the external pacing load for maintaining a conducting state of charge coupled devices that require a high operating current.

Accordingly, therapy delivery circuit 84 may include an internal adjustable load for controlling the current flowing through the high voltage output circuitry to enable cardiac pacing via a low impedance pacing electrode vector, e.g., including coil electrode 24 and/or coil electrode 26 and/or housing 15. In some examples, as described below in conjunction with FIG. 4, therapy delivery circuit 84 includes an internal adjustable load implemented as a current sink controlled to pull current needed to hold charge coupled switches of the high voltage output circuit in a conducting state as needed for delivering cardiac pacing pulses having a pacing voltage amplitude that is relatively low compared to the CV/DF shocks. An ICD operating according to the techniques disclosed herein controls an internal adjustable load to regulate the current required to operate the HV output circuitry in a manner that enables pacing pulse delivery while minimizing operating current drain that is not delivered to the external pacing load. In this way, coil-to-coil or other low impedance pacing electrode vectors can be used for delivering cardiac pacing in an extra-cardiac ICD system while conserving power source 98 and the useful life of the ICD.

In some examples, in addition to being configured to deliver therapeutic electrical stimulation pulses to the patient's heart under the control circuit 80, therapy delivery circuit 84 may be controlled to deliver electrical stimulation pulses for inducing tachyarrhythmia, e.g., T-wave shocks or trains of induction pulses, upon receipt of a programming command from external device 40 (FIG. 1A) by telemetry circuit 88, e.g., during ICD implant or follow-up testing procedures.

Sensor(s) 99 may include one or more sensors for sensing physiological signals for various patient monitoring purposes. For example, sensor(s) 99 may include an accelerometer for sensing a patient physical activity signal for use in controlling the rate of cardiac pacing pulses delivered by therapy delivery circuit 84 during a rate response pacing mode. Examples of other sensors that may be included in ICD 14 include a temperature sensor, oxygen saturation sensor, pH sensor, and heart sound sensor among others.

Telemetry circuit 88 includes a transceiver and antenna for communicating with external device 40 (shown in FIG. 1A) using RF communication or other communication protocols as described above. Control parameters utilized by control circuit 80 for sensing cardiac event signals, detecting arrhythmias, and controlling therapy delivery may be programmed into memory 82 via telemetry circuit 88. Under the control of control circuit 80, telemetry circuit 88 may receive downlink telemetry from and send uplink telemetry to external device 40. Telemetry circuit 88 may receive a pacing voltage amplitude, for example, selected and programmed by a user interacting with external device 40. Therapy control circuit 94 may select the cardiac pacing voltage source and pacing output pathway in accordance with the pacing voltage amplitude and pass control signals to therapy delivery circuit 84 for controlling delivery of pacing pulses by therapy delivery circuit 84 according to the selected pacing parameters.

FIG. 4 is a conceptual diagram of circuitry that can be included in therapy delivery circuit 84 of ICD 14 according to some examples. Therapy delivery circuit 84 includes HV charging circuit 152 configured to charge one or more HV holding capacitors 162 to deliver CV/DF shocks using coil electrode 24, coil electrode 26 and/or housing 15 via HV output circuit 160. HV charging circuit 152, HV holding capacitor 162 (also referred to herein as “HV capacitor” 162), and HV output circuit 160 may be included in the HV therapy circuit 100 shown in FIG. 3.

In response to control circuit 80 detecting a need for CV/DF shock therapy based on an analysis of cardiac electrical signals sensed by sensing circuit 86, HV holding capacitor 162 may be charged to a shock voltage amplitude by HV charging circuit 152 for delivering a CV/DF shock under the control of control circuit 80. HV charging circuit 152 may include a transformer to step up the battery voltage of power source 98 (shown in FIG. 3) in order to achieve charging of HV holding capacitor 162 to a voltage greater than the battery voltage. HV charging circuit 152 may include one or more transformers, switches, diodes, and/or other devices for operating to charge HV holding capacitor 162 to a desired voltage.

Control circuit 80 may pass a charge signal to HV charging circuit 152 to initiate charging and receive feedback signals from the HV charging circuit 152 to determine when HV holding capacitor 162 is charged to a shock voltage amplitude, e.g., corresponding to a programmed CV/DF shock energy, which may be selected based on defibrillation threshold testing or set to a nominal defibrillation energy, e.g., 20 Joules or more. A charge completion signal may be passed from control circuit 80 to HV charging circuit 152 to terminate charging of HV holding capacitor 162 in response to determining that the HV holding capacitor 162 is charged to a desired voltage.

While HV holding capacitor 162 is illustrated as a single capacitor in FIG. 4, it is to be understood that a combination of capacitors may be configured to function as a HV holding capacitor chargeable to a shock voltage amplitude. For example, two or more HV capacitors may be provided in HV therapy circuit 100 having an effective capacitance of 100 to 200 microfarads, or about 140 to 160 microfarads as examples. The HV capacitors may be charged to hold 750 to 800 V, for example, in order to deliver CV/DF shocks having a pulse energy of 20 Joules or more, 30 Joules or more or 40 Joules or more, as examples, though lower energy CV/DF shocks could be delivered when the patient's defibrillation threshold is lower.

A CV/DF shock can be delivered to the heart by discharging HV holding capacitor 162 under the control of control circuit 80 according to signals passed to HV output circuit 160, e.g., via a control bus from therapy control circuit 94 (shown in FIG. 3). HV output circuit 160 includes switching circuitry, which may be in the form of an H-bridge including high side switches 180a-180c and low side switches 182a-182c, that are biased into a conducting state (e.g., switched ON or enabled) from a non-conducting state (e.g., switched OFF or disabled) by signals from therapy control circuit 94 of control circuit 80.

As used herein, “low side” generally refers to the current path from the load to ground (common). For example, a low side switch 182a-c conducts current to ground from an electrode terminal 124, 126 or 115 coupled to a pacing electrode. As used herein, “high-side” generally refers to the current path from the cardiac electrical stimulation voltage source to the load. For example, a high side switch 180a-c conducts current from a selected cardiac pacing voltage source of therapy delivery circuit 84 to an electrode terminal 124, 126 or 115 coupled to a pacing electrode. As further described below, the load can include the internal adjustable load 156 that is coupled to the low side of high side switches 180a-c for pulling current through a high side switch 180a-c to ground. In some instances, the load during cardiac pacing pulse delivery is the external pacing load coupled to electrode terminals 124, 126 and/or 115. In other instances, as further described below, the load is the combination the external pacing load coupled to electrode terminals 124, 126 and/or 115 and the internal adjustable load 156 coupled between the low side of high side switches 180a-c and ground.

High side switches 180a-180c may each include one or more electronic switching devices. In some examples, high side switches 180a-180c may each include an anode gated thyristor (AGT), metal oxide semiconductor field effect transistor (MOSFET), insulated gate bipolar transistor (IGBT), MOS-controlled thyristor (MCT), silicon-controlled rectifier (SCR) or other switching device or combination of switching devices having a high voltage rating. The high side switches 180a-c are generally high voltage rated switches that require a high operating current to bias the switch into a conducting or “on” state from a non-conducting or “off” state such that current leakage from HV holding capacitor 162 can be minimized when high side switches 180a-c are not enabled. High side switches 180a-180c may be charge coupled devices, such as AGTs, that can be controlled without requiring bootstrapping. One or a combination of high side switches 180a-180c is/are switched on by a trigger current signal, e.g., from control circuit 80, and held in a conducting state for conducting current from the HV capacitor 162 to an electrode terminal 124, 126, or 115 coupled to coil electrode 24, coil electrode 26, or housing 15, respectively, selected as the CV/DF cathode electrode. A different one of coil electrode 26, coil electrode 24 or housing 15 may be selected as the return anode electrode by switching on a selected one of low side switches 182a, 182b or 182c, which is coupled to the respective electrode terminal 124, 126, or 115 of the selected anode electrode.

A relatively high current trigger signal may be passed from control circuit 80 to switch a selected high side switch 180a, 180b or 180c to an ON state, to start discharging HV capacitor 162 for shock delivery. During discharging of HV capacitor 162 through a selected shock delivery pathway, the high current flowing through the enabled high side (charge coupled) switch 180a, 180b or 180c holds the switch in the conducting state until the low side switch 182a, 182b, or 182c is switched OFF, to a non-conducting state, by control circuit 80. When the low side switches 182a-182c are switched to a non-conducting state, current flowing through the high side switches 180a-180c is stopped or falls below a specified holding current for high side switches 180a-180c. High side switches 180a-180c are switched OFF in this way, terminating shock delivery. High side switches 180a-c may require a relatively high trigger current from control circuit 80 of 100 to 200 milliamps, for example, to bias the switch into a conducting state.

Low side switches 182a-182c may each include one or more switching devices, which may be implemented as SCRs, IGBTs, MOSFETs, MCTs, and/or other components or combinations of components. A low side switch 182a, 182b or 182c is biased in a conducting state by a control signal from therapy control circuit 94 of control circuit 80 to select a return path through an anode electrode selected from coil electrodes 24 and/or 26 or housing 15. Low side switches 182a-182c can be relatively low impedance switches, to minimize losses during defibrillation, and can be switched to an ON state by a relatively low current control signal, e.g., less than 10 milliamps, from control circuit 80.

High side switches 180a-180c and low side switches 182a-182c are controlled to be ON or OFF by control circuit 80 (e.g., by signals received from therapy control circuit 94 shown in FIG. 3) at the appropriate times for delivering a CV/DF shock. For instance, one of high side switches 180a, 180b or 180c may be switched to an ON state simultaneously with one of low side switches 182a, 182b, or 182c, without switching on both of the “a,” “b” or “c” switches across a given electrode terminal 124, 126 or 115, respectively, at the same time. To deliver a biphasic electrical stimulation pulse using coil electrode 24 and housing 15, for instance, switch 180a and 182c may be switched to ON states to deliver a first phase of the biphasic pulse. Before HV capacitor 162 is fully discharged, switches 180a and 182c are switched to an OFF state after the first phase, and switches 180c and 182a are switched to an ON state to reverse the polarity of the biphasic pulse and deliver the second phase of the biphasic pulse. Switches 180b and 182b remain in an OFF (non-conducting) state in this example when coil electrode 26 is not selected for use in the CV/DF shock delivery vector. In other examples, coil electrode 26 may be included instead of coil electrode 24 or simultaneously selected with coil electrode 24 to function as a cathode electrode or an anode electrode. Examples of circuitry and techniques for delivering a CV/DF shock pulse via HV output circuitry are generally disclosed in U.S. Pat. No. 10,159,847 (Rasmussen, et al.), incorporated herein by reference in its entirety.

When a cardiac pacing pulse is needed and the pacing capture threshold is very high, e.g., greater than 8 V, 10 V, 16 V, 20 V, or 30 V, control circuit 80 may control HV charging circuit 152 to charge HV capacitor 162 to a programmed pacing voltage amplitude, less than the voltage required for CV/DF shock delivery. A relatively high voltage cardiac pacing pulse may be delivered via HV output circuit 160 by applying control signals to enable one or more selected high side switches 180a-c and enable one or more selected low side switches 182a-c during each phase of a cardiac pacing pulse as needed for discharging HV capacitor 162 via a selected pacing electrode vector including coil electrode 24 and/or coil electrode 26 and/or housing 15.

However, the current conducted through the high side switches 180a, 180b and/or 180c to the external pacing load during a cardiac pacing pulse is much lower than the current conducted through the high side switches 180a, 180b and/or 180c during a CV/DF shock pulse due to the lower charge of the cardiac pacing voltage source, e.g., HV holding capacitor 162 charged to a cardiac pacing pulse amplitude. The current conducted through the HV output circuit 160 to the external pacing load when a cardiac pacing voltage source is coupled to HV output circuit 160 can be less than the current required to hold the high side switches 180a, 180b and/or 180c in a conducting state during the cardiac pacing pulse. As described below, internal adjustable load 156 is provided in parallel to the external pacing load, e.g., between the low side of high side switches 180a, 180b and 180c and ground. Internal adjustable load 156 is electrically coupled in parallel to the electrode terminals 124, 126 and 115 that can be coupled to the external pacing load. Internal adjustable load 156 is controlled to pull additional current (in addition to the current flowing to the external pacing load) through an enabled high side switch 180a, 180b or 180c to reduce or eliminate the need for applying a continuous gate current to hold the switch in a conducting state. The current pulled by the adjustable load 156 can be less than a continuous gate current thereby conserving current drain from power source 98 needed for cardiac pacing pulse delivery. In this way, the adjustable load 156 enables cardiac pacing pulses having a relatively low voltage amplitude compared to CV/DF shocks to be delivered via HV output circuit 160 to a low impedance pacing electrode vector, e.g., including coil electrodes 24, 26 and/or housing 15, coupled to respective electrode terminals 124, 126 and 115.

The internal adjustable load 156, also referred to herein as “adjustable load” 156, pulls current from a selected cardiac pacing voltage source to ground through a selected high side switch 180a, 180b, or 180c during each phase of a monophasic, biphasic or multiphase (e.g., triphasic, etc.) pacing pulse. Adjustable load 156 may be a current sink that includes multiple transistors, e.g., multiple field effect transistors (FETs), or other circuit components that can be digitally controlled, e.g., by multi-bit registers, to set the current level that is pulled by the adjustable load 156. The FETs can be prevented from being turned ON by gate switching during CV/DF shock delivery (by ICD 14 or another internal or external device) or at other times that additional current pulled through the high side switches 180a-c is not needed or undesirable. A ballasting resistor may be included on the drain of each FET in the adjustable load 156 to aid in regulating the current drawn and avoid overcurrent.

In an example, adjustable load 156 may include a programmable current mirror for setting a reference current with a gain stage for amplifying the reference current. The reference current may be 10, 20 or 30 microamperes, for example, with a gain stage amplifying the reference current by 1000× to draw a current of 10, 20 or 30 mA for instance through a high side switch 180a, 180b or 180c to ground during delivery of a cardiac pacing pulse to the external pacing load (via electrode terminals 124, 126 and/or 115). While a current mirror with a gain stage is one example of a current sink circuit that may be implemented in the internal adjustable load 156, other current sink circuits may be used. In other examples, internal adjustable load 156 may be implemented using a programmable resistor bank or a programmable current source including an amplifier/transistor pair with a digital-analog-converter input for providing an amplifier reference.

Adjustable load 156 may be digitally controlled to pull current between 5 milliamperes (mA) and 150 mA, between 15 mA and 120 mA or between 20 mA and 100 mA as non-limiting examples. To illustrate, adjustable load 156 may be digitally controlled to pull 10 mA, 20 mA, 30 mA, 40 mA, 50 mA, 60 mA, 80 mA, or 100 mA. It is to be understood that in a given ICD, different ranges and step sizes of the controlled adjustable current drawn by internal adjustable load 156 may be made available based on the available programmable cardiac pacing pulse amplitudes, the expected pacing electrode vector impedance, required latching and holding currents of high side switches 180a-c and other factors. A digital register may store values, which may be programmable by firmware or software, that control a voltage reference input to a current sink gain stage. In other examples, the digital register may store programable values that control legs of a current source for adding specific amounts of current to the total current pulled by the adjustable load 156. In this situation, the register can be set to a specific output current value. In still other examples, the digital register may control switches that change the value of a resistor on the source of an output transistor on the output of the current sink gain stage. Thus, the adjustable load 156 can be controlled by control circuit 80, e.g., via a digital register, to pull a specified current during each pacing pulse, and during each phase of a pacing pulse, as further described below.

To initiate a pacing pulse, control circuit 80 may pass a trigger current signal to switch a selected one (or more) of high side switches 180a, 180b and/or 180c to an ON state. The selected one (or more) of high side switches 180a, 180b and/or 180c is/are coupled to a cathode electrode (or combination of cathode electrodes), selected from coil electrode 24, coil electrode 26 and/or housing 15 in this example. The pacing pulse may be initiated upon expiration of a pacing escape interval, e.g., a lower rate interval, a hysteresis interval, an asystole detection interval, a post-shock pacing interval, or an ATP interval. The pacing interval may be timed out by timing circuit 90 or by timers included in therapy control circuit 94. In some instances, control circuit 80 may initiate delivery of a cardiac pacing pulse signal in response to detecting a pace triggering event, e.g., a sensed R-wave for synchronizing a leading pacing pulse of an ATP sequence or for triggering a back-up safety pacing pulse.

Control circuit 80 may selectively control the current drawn by adjustable load 156 based on the cardiac pacing pulse voltage amplitude. For a given pacing electrode vector impedance, which can be referred to as the “external load” or “external pacing load,” the adjustable load 156 may be controlled to draw a higher current when the pacing pulse voltage amplitude is relatively lower. The adjustable load 156 may be controlled to draw a relatively lower current when the pacing pulse voltage amplitude is relatively higher. In this way, the current drawn by the internal adjustable load 156 can be minimized to avoid unnecessary current drain from power source 98 while avoiding failed delivery of a cardiac pacing pulse or premature truncation of a cardiac pacing pulse due to the current flow through high side switch(es) 180a, 180b and/or 180c falling below the current required to hold the high side switches in a conducting state.

Control circuit 80 may control the adjustable load 156 to cause a first current flow, which may be referred to as a “latching current” through one or more selected high side switches 180a, 180b and/or 180c to hold the selected switch(es) in a conducting state during a first portion of a phase of the cardiac pacing pulse. The latching current can be sustained during a latch period applied during the first portion of a phase of the cardiac pacing pulse. The cardiac pacing pulse may be a monophasic, biphasic, triphasic or other multi-phasic pulse. Each phase may be defined by a phase duration. The total cardiac pacing pulse width is the total of the phase durations of a multiphasic pacing pulse. A latch period may be applied for pulling current by adjustable load 156 during the first portion of a monophasic pulse. A latch period may be applied for pulling current by adjustable load 156 during the first portion of the first phase and/or the first portion of the second phase of a biphasic pulse. A latch period may be applied for pulling current by adjustable load 156 during one or more phases of a multiphasic cardiac pacing pulse. The latching current may be up to a maximum current required to maintain a selected high side switch 180a, 180b or 180c in the ON state during the latch period immediately after a trigger current signal has been removed that turns the high side switch to an ON state from an OFF state. The latching current pulled by the adjustable load 156 plus the current flowing to the external pacing load together equal a total current flowing through the enabled high side switch 180a, 180b or 180c that is sufficient to maintain the high side switch in a conducting state just after the trigger current is removed. This total current is at least a specified minimum latching current of the high side switch.

Control circuit 80 may control the adjustable load 156 to pull a second current, which may be referred to as a “holding current,” to maintain an enabled high side switch 180a, 180b and/or 180c in a conducting state from the expiration of the latch period to the expiration of the phase duration of the given phase of the cardiac pacing pulse. The expiration of the phase duration may coincide with the expiration of the pacing pulse width. The holding current may be up to a maximum current required to maintain the switch in a conducting state after the latch period. The holding current pulled by the adjustable load 156 plus the current flowing to the external pacing load together equal a total current flowing through the high side switch 180a, 180b or 180c sufficient to hold the high side switch in a conducting state. This total current is at least a specified minimum holding current of the high side switch. When current flowing through a high side switch 180a, 180b or 180c falls below the minimum required holding current, the high side switch turns OFF to a non-conducting state. If the latching current or the holding current pulled by the internal adjustable load in combination with the external load current falls below the current required to maintain the high side switch 180a, 180b or 180c in a conducting state, the cardiac pacing pulse may be truncated prematurely and may fail to capture the myocardial tissue. The current flow through the internal adjustable load can be controlled by control circuit 80 to maintain the charge coupled, high side switch 180a, 180b or 180c in a conducting state, taking into account the external load current so that the internal adjustable load current can be minimized to conserve power source 98. By minimizing the internal adjustable load current to achieve a total current (internal adjustable load current plus external pacing load current) that meets the specified latching and holding currents of the high side switch 180a, 180b or 180c, the decay rate of the charge on the HV capacitor 162 can be minimized, thereby minimizing the voltage decay rate of the delivered pacing pulse. In this way, the pulse energy delivered to the pacing electrodes for achieving pacing capture can be maximized (for a given starting pacing pulse amplitude) while still pulling enough current through the high side switch 180a, 180b or 180c to keep the switch in a conducting state during a given phase of the cardiac pacing pulse.

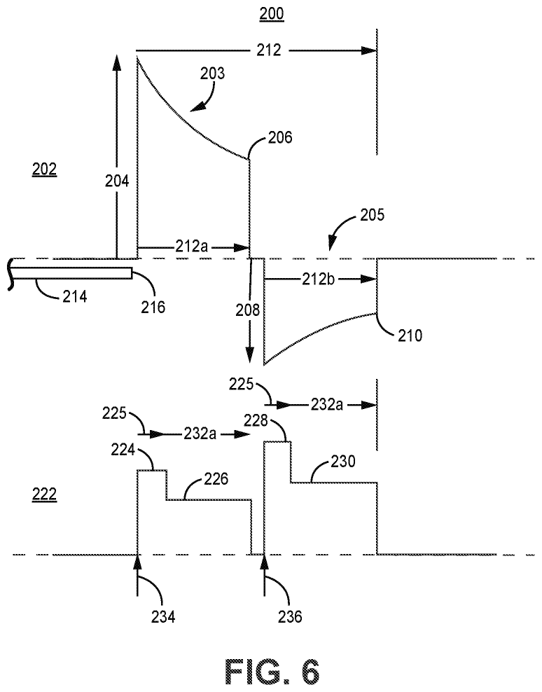

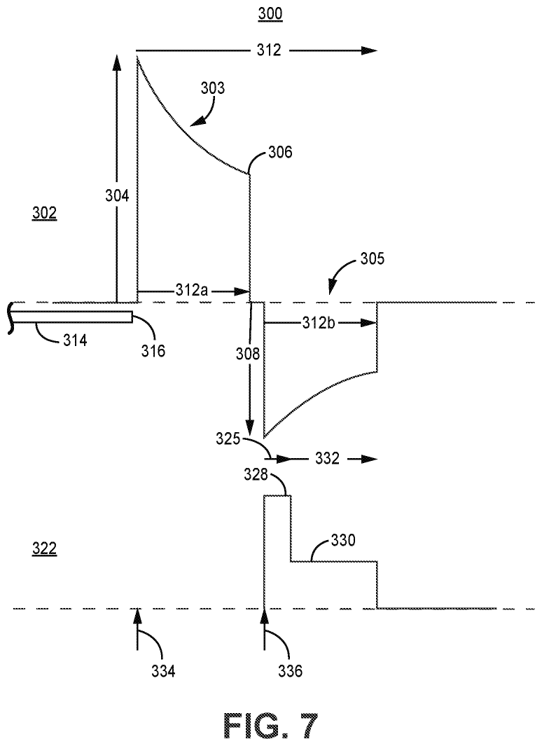

In some examples, the holding current pulled by adjustable load 156 during a second portion of a cardiac pacing pulse phase may be less than the latching current. Because less current can be required for maintaining the high side switches 180a-c in a conducting state after the latch period, power source 98 can be conserved by controlling adjustable load 156 to decrease the current pulled after the latch period. As further described below in conjunction with FIG. 6, the adjustable load 156 may be controlled by therapy control 94 of control circuit 80 to apply a different latching current and a different holding current for each phase a biphasic or multiphasic cardiac pacing pulse. In some examples, e.g., as described below in conjunction with FIG. 7, adjustable load 156 is disabled by control circuit 80 during a first phase of a biphasic or multiphasic pacing pulse so that all current flows to the external pacing load. Control circuit 80 may control adjustable load 156 to pull a latching current and a holding current during a second or later phase of a multiphasic pacing pulse when the cardiac pacing voltage source has been partially discharged during the first, earliest phase of the multiphasic pacing pulse.

When a pacing pulse width expires, e.g., as determined by therapy control circuit 94 of control circuit 80 or by a timer included in therapy delivery circuit 84, the low side switch 182a, 182b or 182c can be turned OFF by a control signal to stop the flow of current to the external pacing load, and the adjustable load 156 may be disabled. When adjustable load current and the external pacing load current are stopped, the current flowing through high side switches 180a, 180b and/or 180c falls below the current needed to hold the high side switches in a conducting state. The high side switches 180a-c are thereby switched OFF, terminating the cardiac pacing pulse.

In some examples, housing 15 is used as an active can electrode only during CV/DF shock delivery. In this case, cardiac pacing pulses delivered when the internal adjustable load 156 is enabled are delivered via a pacing electrode vector between coil electrodes 24 and 26. In other examples, housing 15 may be available for use as a return anode with either or both of coil electrodes 24 and 26 selected as the cathode electrode. In still other examples, housing 15 may be available as the pacing cathode electrode with either or both of coil electrodes 24 and 26 selected as the return anode electrode. The pacing pulse is delivered via a selected pacing cathode electrode(s) by selectively triggering and holding the high side switch 180a, 180b and/or 180c coupled to the respective electrode terminal 124, 126 or 115 that is in electrical contact with the cathode electrode(s) during a first phase of the cardiac pacing pulse.

If the pacing pulse is a biphasic pacing pulse, the polarity of the pacing pulse may be reversed by triggering a different high side switch(es) 180a, 180b or 180c coupled to the respective electrode terminal(s) 124, 126 or 115 that is in electrical contact with the anode electrode(s) during a second phase of the pacing pulse. The anode electrode(s) is/are coupled to ground via low side switch(es) 182a, 182b and/or 182c during a first phase of the cardiac pacing pulse. If a biphasic pacing pulse is being delivered, the cathode electrode(s) is/are coupled to ground via a low side switch(es) 182a, 182b and/or 182c during the second phase of the cardiac pacing pulse. A multiphasic pacing pulse may be delivered by controlling high side switches 180a-c and low side switches 182a-c as needed for discharging a cardiac pacing voltage source to the external pacing load during each phase of the multiphasic pacing pulse.

FIG. 5 is a conceptual diagram of therapy delivery circuit 84 according to another example. In FIG. 4, the HV capacitor 162 charged to a pacing voltage amplitude by HV charging circuit 152 may be coupled to HV output circuit 160 as the cardiac pacing voltage source. In various examples, one or more cardiac pacing voltage sources may be available for generating the cardiac pacing pulse that is delivered via the electrode terminals 124, 126 and/or 115. The cardiac pacing voltage source is not necessarily limited to being the HV capacitor 162 charged to the pacing voltage amplitude. As shown in FIG. 5, a cardiac pacing voltage source that may be coupled to HV output circuit 160 for delivering a cardiac pacing pulse according to the techniques disclosed herein may include a voltage regulator 154 in some examples. Additionally or alternatively, a cardiac pacing voltage source that may be coupled to HV output circuit 160 for delivering a cardiac pacing pulse may include one or more holding capacitors 142 and 146 charged to a multiple of the battery of power source 98 by a charge pump 134. In this example, the cardiac pacing voltage source may be selectable between the HV holding capacitor 162, the output of voltage regulator 154 (if present) and/or holding capacitors 142 and/or 146, which may be included in the LV therapy circuit 102 (see FIG. 3).

In some examples, voltage regulator 154 may be configured to pass a voltage output signal to HV output circuit 160 for delivering the cardiac pacing pulse via electrode terminals 124, 126 and/or 115. Charging of HV capacitor 162 by HV charging circuit 152 may be controlled by control circuit 80 to produce a rail voltage, e.g., 10 to 50 V or about 20 to 40 V as examples, for providing a positive DC voltage that can be used to power various components of therapy delivery circuit 84. Voltage regulator 154 may receive the rail voltage and provide a voltage regulated output signal having a desired cardiac pacing pulse voltage amplitude, which may be stepped down from the rail voltage, to HV output circuit 160. For example, HV charging circuit 152 may be controlled by control circuit 80 to charge the HV capacitor 162 to 16 V, 18 V, 20 V, 30 V, 40 V, 50 V or higher to generate a rail voltage that is at least equal to or greater than a desired cardiac pacing pulse voltage amplitude. Voltage regulator 154 may be configured to regulate the rail voltage to a programmed pacing pulse voltage amplitude, e.g., 15 to 30 V or about 16 to 20 V as examples. In some examples, voltage regulator 154 may be configured to set an output voltage to a fixed value, e.g., 16 to 18 V, that is passed to HV output circuit 160 when a voltage source selection switch 155 is coupled to the output of voltage regulator 154. Voltage source selection switch 155 may be a 3-position switch for coupling HV capacitor 162 to HV output circuit 160 in one position, coupling voltage regulator 154 to HV output circuit 160 in a second position, or open in a third position. In other examples, voltage regulator 154 may receive a control signal from control circuit 80 for adjusting the amplitude of the output voltage signal to a programmed pacing pulse voltage amplitude.

In some examples, a cardiac pacing voltage source may include one or more holding capacitors 142 and 146 that can be charged to a pacing voltage amplitude by a charge pump 134. The holding capacitors 142 and 146 are referred to herein as “low voltage” (LV) holding capacitors because HV capacitor 162 may be a higher rated voltage capacitor that is chargeable to relatively much higher voltages for delivering CV/DF shocks. The LV holding capacitors 142 and 146 may be coupled to HV output circuit 160 via switches 165a and 165b when switch 155 is in an open position for delivering a cardiac pacing pulse via electrode terminals 124, 126 and/or 115 coupled to the respective coil electrode 24, coil electrode 26 and housing 15. It is recognized that more or fewer switches may be included than the switches 155, 165a, 165b for controlling which cardiac pacing voltage source is coupled to the HV output circuit 160 for delivering a pacing pulse. Furthermore, any switches implemented for coupling an alternative cardiac pacing voltage source (other than HV capacitor 162, for example) for delivering cardiac pacing pulses via the HV output circuit 160 may be implemented to withstand the relatively high voltage of the generated cardiac pacing pulse and introduce relatively low impedance in the pacing output circuitry. Control circuit 80 may select a cardiac pacing voltage source from HV capacitor 162, voltage regulator 154 or LV holding capacitors 142 and/or 146 by controlling switches 155, 165a and 165b for connecting a selected cardiac pacing voltage source to the HV output circuit 160. As further described below, a cardiac pacing voltage source may be selected by control circuit 80 based on a pacing capture threshold, programmed pacing pulse amplitude, the maximum pulse amplitude that can be generated by the cardiac pacing voltage source, the type of cardiac pacing therapy being delivered, or other factors.

As described above, therapy delivery circuit 84 may include an LV therapy circuit 102 (see FIG. 3). LV therapy circuit 102 may include an LV charging circuit 132 and an LV output circuit 140. The LV charging circuit 132 may include one or more charge pumps 134 for charging LV holding capacitors 142 and/or 146 to a pacing pulse amplitude. Charge pump 134 may charge LV holding capacitors 142 and/or 146 up to a multiple of the battery voltage of power source 98. The charge pump 134 may be referred to as an “Nx” charge pump because it may be capable of charging LV holding capacitors 142 and 146 up to N times (Nx) the battery voltage of power supply 98, where N may be equal to any selected multiple of the battery voltage, e.g., up to two, three, four, five or six times the battery voltage, as examples. A state machine of control circuit 80 may control charging of LV holding capacitors 142 and/or 146 to a programmed pacing pulse amplitude using a multiple of the battery voltage of power source 98. LV holding capacitors 142 and 146 may each have a capacitance of 50 microfarads or less or as low as 10 microfarads or less, as examples.

In some instances, one of ring electrodes 28 or 30 may be selected as the pacing cathode electrode for delivering cardiac pacing pulses. A capacitor selection switch 143 or 147 may be biased to a conducting state by a control signal from control circuit 80 for charging a selected LV holding capacitor 142 or 146 by a charge pump 134 to achieve a desired pacing pulse amplitude in a lower range of pacing pulse amplitudes. The charged holding capacitor 142 or 146 may be discharged via a tip capacitor 145 or 149, respectively, by switching on an electrode selection switch 144 or 148 after charge completion to deliver a pacing pulse to a selected cathode electrode, e.g., ring electrode 28 in electrical contact with electrode terminal 128 or ring electrode 30 in electrical contact with electrode terminal 130. The other ring electrode 30 or 28 may serve as the return anode electrode.

However, when the pacing electrode vector includes coil electrode 24, coil electrode 26, and/or housing 15, control circuit 80 may enable one or both of voltage source selection switches 165a and/or 165b to conduct the cardiac pacing pulse signal from LV holding capacitors 142 and/or 146 via switches 165a and/or 165b to the respective electrode terminal 124, 126 or 115 via HV output circuit 160. A voltage source selection switch 155 may be opened by control circuit 80 when the LV holding capacitors 142 and/or 146 are selected as the cardiac pacing voltage source. One of low side switches 182a, 182b or 182c is switched to an ON state to provide a return path from a selected pacing anode electrode, e.g., coil electrode 24, coil electrode 25 or housing 15 that is not used as the cathode electrode. Control circuit 80 may select (or a user may program) a cardiac pacing electrode vector that includes coil electrode 24 and/or coil electrode 26. The LV output circuit 140 may pass a cardiac pacing pulse signal via one or both of switches 165a and 165b in a lower range of pacing voltage amplitudes to HV output circuit 160 for delivering a cardiac pacing pulse via at least one or both of coil electrodes 24 and 26 (and/or housing 15 in some examples). Control circuit 80 may control adjustable load 156 to draw current needed to hold the enabled high side switches 180a-c in a conducting state during each phase of the cardiac pacing pulse.

In some examples, ICD 14 may be configured to deliver cardiac pacing pulses using coil electrodes 24 and/or 26 in a selected one of an upper range, an intermediate range and/or a lower range of pacing pulse amplitudes, e.g., based on the cardiac pacing capture threshold or the results of a pacing capture test. Control circuit 80 can select the HV holding capacitor 162 as a cardiac pacing voltage source when the cardiac pacing pulse amplitude is in an upper range. Control circuit 80 may select the cardiac pacing voltage source by controlling HV charging circuit to charge HV holding capacitor 162 to the pacing pulse amplitude in the upper range, e.g., greater than 16 V, greater than 20 V, greater than 30 V or greater than 40 V, and control HV output circuit 160 to deliver cardiac pacing pulses having an upper range voltage amplitude using the H-bridge switching circuitry of output circuit 160. In some cases, when HV holding capacitor 162 is charged to a pulse amplitude in the upper range, control circuit 80 disables internal adjustable load 156. Discharge of the HV holding capacitor 162 through the external pacing load may result in sufficient current flow through high side switches 180a-c without requiring additional current pulled by adjustable load 156. In other examples, depending at least on the pacing pulse amplitude, control circuit 80 may enable adjustable load 156 to draw current needed to hold the selected high side switches 180a-c in a conducting state during discharging of HV holding capacitor 162 for delivery of a cardiac pacing pulse.

When the pacing pulse amplitude is less than the upper range and falls into an intermediate range, control circuit 80 may select an intermediate pacing voltage source by controlling HV charging circuit 152 to charge HV holding capacitor 162 to an intermediate voltage to generate a rail voltage. The generated rail voltage enables voltage regulator 154 to pass a voltage signal to the HV output circuit 160 for delivering a cardiac pacing pulse having a voltage amplitude in an intermediate range, less than the upper range, via at least one of coil electrode 24 and/or 26 and/or housing 15. The intermediate range of pacing pulse amplitudes may be up to a maximum voltage amplitude available from voltage regulator 154, which may be up to 16 V, up to 18 V, up to 20 V, up to 30 V, or up to 40 V as examples. Control circuit 80 may control the adjustable load 156 to draw the current needed for latching and holding selected ones of the high side switches 180a-c during each phase of the cardiac pacing pulse for generating the intermediate voltage amplitude pacing pulse. During a cardiac pacing pulse having an amplitude in the intermediate range, the current drawn by adjustable load 156 can be higher than the current drawn, if any, by adjustable load 156 during a cardiac pacing pulse having a voltage amplitude in the upper range.

The voltage regulator 154 can be used to generate cardiac pacing pulses in an intermediate voltage range when the pacing capture threshold is greater than the maximum voltage amplitude available from LV therapy circuit 102 (FIG. 3) but not greater than the voltage amplitude available from voltage regulator 154. When the pacing capture threshold is greater than the voltage amplitude available from the voltage regulator 154, the HV therapy circuit 100 may deliver the pacing pulses in the upper range via HV output circuit 160 using HV capacitor 162 charged to the pacing pulse amplitude as the cardiac pacing voltage source.

When the pacing capture threshold is in a lower range, the cardiac pacing voltage source can be the LV therapy circuit 102, e.g., one or more LV capacitors 142 and/or 146 charged to the pacing pulse amplitude by charge pump 134. Control circuit 80 may select the cardiac pacing voltage source by controlling LV charging circuit 132 to charge a LV holding capacitor 142 and/or 146 up to a maximum pulse amplitude available from LV therapy circuit 102, e.g., up to 8 V, up to 10 V, up to 12 V or up to 16 V as non-limiting examples. Control circuit 80 may enable one or both of switches 165a and/or 165b for conducting the lower range cardiac pacing pulse signal to the HV output circuit 160. As described herein, the adjustable load 156 may be controlled by control circuit 80 to draw current to hold selected ones of high side switches 180a-c for delivering the cardiac pacing pulses having a pulse amplitude in the lower range via the HV output circuit 160.

During a cardiac pacing pulse having a lower range voltage amplitude, control circuit 80 may control adjustable load 156 to draw a current that is higher than the current drawn during cardiac pacing pulses having a voltage amplitude in the intermediate range and upper range. The current drawn by adjustable load 156 may include a first, higher latching current followed by a second, lower holding current during each phase of a cardiac pacing pulse to hold a high side switch 180a, 180b or 180c in a conducting state throughout the phase duration of a given phase of the cardiac pacing pulse.

It is to be understood that in some examples, a lower and an upper range of pacing pulse amplitudes may be available for cardiac pacing via the HV output circuit 160 and a pacing electrode vector that includes at least one low impedance, coil electrode 24, coil electrode 26 and/or housing 15 instead of the three lower, intermediate and upper ranges of pacing pulse amplitudes described here. The cardiac pacing voltage source may be selected as voltage regulator 154 for lower range pacing pulse amplitudes, and the cardiac pacing voltage source may be selected as the HV holding capacitor 162 for upper range pacing pulse amplitudes. In other examples, the pacing voltage source may be selected as the voltage regulator 154 for upper range pacing pulse amplitudes. LV holding capacitor(s) 142 and/or 146 may be selected as the pacing voltage source for lower range pacing pulse amplitudes. In still other examples, the pacing voltage source may be selected as the HV holding capacitor 162 for upper range pacing pulse amplitudes or the LV holding capacitor(s) 142 and/or 146 for lower range pacing pulse amplitudes.

LV therapy circuit 102 may be configured for generating both lower range pacing pulse amplitude signals and intermediate range pacing pulse amplitude signals in some examples. For instance, charge pump 134 may include one or more charge pumps for generating cardiac pacing pulse signals. A first charge pump may be used for charging an LV holding capacitor 142 or 146 to a pacing voltage amplitude in a lower range, e.g., up to 8 V, up to 10 V or up to 12 V as examples, which may be delivered via LV output circuit 140, when cardiac capture can be achieved by the relatively low voltage pacing pulses. A single one of LV holding capacitors 142 and 146 may be charged for generating a cardiac pacing pulse having a lower range pacing pulse amplitude.

When an intermediate range pacing voltage amplitude is needed, LV charging circuit 132 may be controlled by control circuit 80 to charge one or both of LV holding capacitors 142 and 146 to a voltage in an intermediate range of the pacing pulse voltage amplitudes, e.g., between 8 V and 30 V or between 10 V and 30 V or between 10 V and 20 V or between 10 V and 16 V as examples, with no limitation intended. One or both of LV holding capacitors 142 and 146 may be charged by the output of a second charge pump included in charge pump 134 in some examples. For instance, the output of the second charge pump may charge an LV holding capacitor 142 or 146 to a multiple of the output of the first charge pump. In an illustrative example, a first charge pump may be a 3× charge pump and a second charge pump may be a 2× charge pump to provide a pacing voltage signal up to 6 times the battery voltage of power source 98 (shown in FIG. 3). In this example, it is to be understood that at least one of LV holding capacitors 142 and 146 has a voltage rating to withstand the higher voltages of the intermediate range.

While two LV holding capacitors are shown FIG. 5, it is to be understood that LV therapy circuit 102 may include, one, two, three or more holding capacitors, which may be selected singly or in various series and/or parallel combinations for generating a cardiac pacing pulse. Each holding capacitor can be provided with the necessary voltage rating needed to withstand the voltages to be stored for generating cardiac pacing pulses in a lower range and, in some examples, one or more intermediate ranges. Furthermore, while LV therapy circuit 102 and LV holding capacitors 142 and 146 are referred to herein as “low voltage” or “LV,” the LV therapy circuit 102 and LV holding capacitors 142 and 146 are not limited to generating cardiac pacing pulses in a lower range as made apparent by the foregoing examples. The term “low voltage” is used to distinguish the maximum pacing pulse voltage amplitude capacity of LV charging circuit 132 for functioning as a cardiac pacing voltage source from the maximum voltage amplitude capacity of HV charging circuit 152 for functioning as a CV/DF shock pulse voltage source.

As such, LV therapy circuit 102 may be used for generating cardiac pacing pulses that may be in a relatively lower range of voltage amplitudes of an overall range of available, programmable cardiac pacing pulse voltage amplitudes that can be generated by therapy delivery circuit 84. It is to be understood, however, that when the pacing pulses are delivered via a low impedance pacing electrode vector, e.g., including any of coil electrode 24, coil electrode 26 and/or housing 15, a relatively high capacitance is generally required in order to maintain an effective pulse amplitude for delivering enough energy to capture the myocardial tissue before the pulse amplitude decays below the capture threshold. In some examples, the relatively lower capacitance of LV holding capacitors 142 and 146 may result in a pacing pulse delivered via a low impedance pacing electrode vector that decays too fast to effectively deliver a pacing pulse that captures the heart. As such, when LV therapy circuit 102 is included in therapy delivery circuit 84, LV holding capacitors 142 and 146 may be used in a cardiac pacing voltage source that is coupled to LV output circuit 140 for delivering cardiac pacing pulses via terminals 128 and 130 but may not be used as a cardiac pacing voltage source that is coupled to HV output circuit 160 in some examples.

LV holding capacitors 142 and 146 and voltage regulator 154 are shown in FIG. 5 as illustrative examples of alternative cardiac pacing voltage sources that may be conceived for use in conjunction with HV output circuit 160 including adjustable load 156 for delivering cardiac pacing pulses via a low impedance pacing electrode vector, e.g., including coil electrode 24, coil electrode 26 and/or housing 15 according to the techniques disclosed herein. It is to be understood that the HV capacitor 162 chargeable to a CV/DF shock amplitude is one cardiac pacing voltage source that may be coupled to HV output circuit 160 for delivering cardiac pacing pulses according to the techniques disclosed herein that include controlling an internal adjustable load current for maintaining selected charge-coupled high side switches 180a-c in a conducting state during each cardiac pacing pulse. It is to be understood, however, that other cardiac pacing voltage sources may be included in therapy delivery circuit 84 for generating cardiac pacing pulses that can be delivered to electrode terminals 124, 126 and/or 115 via HV output circuit 160 using adjustable load 156 for maintaining high side switches 180a-c in a conducting state as needed.

FIG. 6 is a diagram 200 of a cardiac pacing pulse 202 and the current that may be drawn by internal adjustable load 156 during the cardiac pacing pulse according to some examples. For sake of illustration, a biphasic cardiac pacing pulse 202 is shown in FIG. 6. Pacing pulse 202 has a starting pulse amplitude 204 corresponding to the programmed pacing pulse amplitude. With continued reference to the therapy delivery circuitry shown in FIGS. 4 and 5, the cardiac pacing voltage source may be the HV holding capacitor 162 charged to the voltage of pulse amplitude 204, which may be in an upper range of cardiac pacing pulse amplitudes. The cardiac pacing voltage source may include HV holding capacitor 162 charged for generating a rail voltage received by the voltage regulator 154. A voltage regulated signal may be passed from voltage regulator 154 to HV output circuit 160 having a voltage equal to (or slightly greater than) the pulse amplitude 204. In other instances, the cardiac pacing voltage source may be one or more LV holding capacitors 142 and/or 146 charged to (or slightly greater than) the voltage of pulse amplitude 204, e.g., in a lower range of pacing pulse amplitudes.

Pacing pulse 202 may have a total pulse width 212 defined by the duration 212a of the first phase 203 and the duration 212b of the second phase 205. A negligible time delay between the first phase 203 and the second phase 205 may occur when the switches of the H-bridge of HV output circuit 160 are switched to reverse the polarity of the second phase 205 of cardiac pacing pulse 202. In the example shown, the first phase duration 212a and the second phase duration 212b are shown to be equal, but each phase duration 212a and 212b could be different from the other in some examples. The cardiac pacing pulse 202 decays exponentially from the starting pulse amplitude 204 to an ending amplitude 206 of the first phase 203 due to the holding capacitor(s) of the selected voltage source, e.g., HV capacitor 162, being discharged through the external pacing load over the first phase duration 212a.

The second phase 205 has a starting amplitude 208 corresponding to the ending amplitude 206 of the first phase. The holding capacitor(s) providing the voltage signal for generating the pacing pulse 202 continue to discharge during the second phase 205. The starting amplitude 208 of the second phase 205 exponentially decays to the ending, amplitude 210 at the expiration of the pacing pulse width 212.

Pacing pulse 202 may be started at the expiration 216 of a cardiac pacing interval 214. Upon detecting the expiration 216 of pacing interval 214, control circuit 80 may apply a trigger current 234 to a selected one of high side switches 180a, 180b or 180c to turn the switch ON from an OFF state. When the trigger current is removed during a high voltage CV/DF shock, the high current flow through a high side switch maintains the switch in a conducting state. However, when the trigger current 234 is removed during a cardiac pacing pulse, the current flow through the high side switch may be too low to maintain the switch in a conducting state. The cardiac pacing pulse could be truncated prematurely and may fail to capture the cardiac tissue for causing a depolarization and pacing evoked response. Applying the trigger current 234 throughout each phase of the cardiac pacing pulse to hold a high side switch 180a, 180b or 180c in a conducting state results in excess current drawn from the ICD power source 98, which can lead to a premature end of the functional life of the ICD. Applying the trigger current 234 throughout each phase of the cardiac pacing pulse may lead to a faster decay rate of the pacing pulse amplitude which could result in a loss of pacing capture.

In order to deliver a pacing pulse 202 that is not prematurely truncated before the expiration of the pacing pulse width 212 and minimize wasted or excessive current drain of the ICD power source, control circuit 80 may control the internal adjustable load 156 to pull a current signal 222 through the selected high side switch 180a, 180b or 180c during biphasic pacing pulse 202 to maintain the selected high side switches in a conducting state throughout each respective phase 203 and 205.

A first high side switch 180a, 180b or 180c may be latched in the conducting state by a latching current 224 pulled by the adjustable load 156 for a latch period 225 at the start of the first phase 212a of pacing pulse 202. The adjustable load 156 is controlled by control circuit 80 to pull the first latching current 224 from the time that the trigger signal 234 is removed for a specified latch period 225 to hold the high side switch ON immediately after the trigger signal 234 is removed. The latch period 225 may be 50 to 300 microseconds long, for example, and is 120 microseconds long in an example. The latch period may be a fixed value in some examples. In other examples, the latch period may be adjustable, e.g., programmable or adjusted by control circuit 80. In some cases, the high side switches 180a-c may require a lower latching current when a longer latch period is applied. In this case, an overall reduced current drain may be achieved by using a long latch period. The latch period may be adjusted depending on components used in HV output circuit 160, the pacing pulse amplitude, and external load impedance among other factors. As described above, the adjustable load 156 can be a current sink that is digitally controlled to pull a specified current during the latch period 225 and after the latch period 225 for the duration of a given phase of the pacing pulse.

The adjustable load 156 is controlled to pull a constant current that has a constant impact on the pacing pulse decay profile, independent of the instantaneous voltage amplitude. In contrast, an internal resistor that could be provided as a “current shunt” in parallel to the external pacing load to pull additional current through a high side switch 180a, 180b or 180c will shunt a non-constant current that will be a relatively higher current at the leading peak voltage of the pacing pulse and a relatively lower current at the ending, trailing voltage of the pacing pulse. The shunted current through an internal resistor is directly proportional to the instantaneous voltage amplitude of the pacing pulse thus having a greater impact on the capacitor charge decay rate at the start of the pulse and overall greater impact on the cardiac pacing pulse decay profile. The constant latching current and constant holding current (described below) pulled by the digitally programmable adjustable load as described herein causes less pacing pulse signal distortion and can minimize any loss of delivered pacing energy, particularly at the starting pulse amplitude and early in the pacing pulse when the instantaneous voltage is highest.

Upon expiration of the latch period 225, control circuit 80 controls the adjustable load 156 to draw a holding current 226 that can be less than the latching current 224. The holding current 226 prevents the selected high side switch 180a, 180b or 180c from turning to an OFF state prematurely, prior to the expiration of the first phase duration 212a of pacing pulse 202. The holding current 226 is pulled for a time period 232a extending from the expiration of the latch period 225 to the expiration of the first phase duration 212a. The holding current required to prevent the selected high side switch 180a, 180b or 180c from turning off after the latch period 225 expires is generally lower than the latching current required to hold the switch in a conducting state immediately after the trigger current 234 is removed. By pulling a lower holding current 226 for the remaining portion 232a of the first phase duration 212a than the latching current 224 that is drawn during the latch period 225, the ICD power source can be conserved.

The latching current 224 and the holding current 226 pulled during the first phase 203 of pacing pulse 202 by the internal adjustable load 156 may be selectively controlled by control circuit 80 based on an expected external load current and the specified latching and holding currents required to maintain the selected high side switch 180a, 180b or 180c in a conducting state. The external load current flowing through the high side switch 180a, 180b or 180c depends on the pacing pulse amplitude 204 and the pacing load impedance.

In an illustrative example, if the programmed pacing pulse amplitude corresponding to starting pulse amplitude 204 is 10 V and the external pacing load impedance (e.g., the pacing electrode vector impedance) is 250 ohms, the external load current may be 40 mA (I=V/R or 10 V divided by 250 ohms). If the specified latching current required to hold the high side switch in an ON state during the latch period 225 is 120 mA, the latching current 224 during first phase 203 can be 80 mA (120 mA minus 40 mA). The specified latching current (e.g., according to manufacturer specification) needed to hold a high side switch in an ON state may be between 70 and 150 mA or between 80 and 120 mA in various examples. The latching current pulled by the internal adjustable load 156 may be up to the specified current required to hold the high side switch in a conducting state according to manufacturer specification but may be minimized by taking into account the external load current. The total of the latching current pulled by the internal adjustable load 156 and the external pacing load current can be at least the specified current required during the latching period to hold the high side switch 180a, 180b or 180c in a conducting state.

In some examples, the external pacing load impedance may be assumed to be a default impedance corresponding to the selected pacing electrode vector so that the latching current 224 may be determined by control circuit 80 based on pacing pulse amplitude 204. The default pacing electrode vector impedance and a specified required latching current for the implemented high side switch may be known values that are not expected to change. Because the default pacing electrode vector impedance and the specified required latching current for the high side switch can be assumed to be fixed values, control circuit 80 may select the adjustable load latching current 224 and the adjustable load holding current 226 of the first phase 203 based on the programmed starting pulse amplitude 204. In some examples, the holding current 226 pulled during the pacing pulse first phase 203 and/or holding current 230 pulled during the second phase 205 may be selected based on a required holding current specified for the high side switches 180a-c.

Table I below lists the latching currents and holding currents that may be selected by control circuit 80 to be drawn by internal, adjustable load 156 during the latch period 225 and the holding period 232a of the first phase 203 of pacing pulse 202 based on the pulse amplitude 204. The latching currents and holding currents listed in Table I may be established based on an assumed 250 ohm external pacing load impedance (e.g., pacing electrode vector impedance) and a specified required current of 120 mA during a latch period for the implemented high side switches 180a-c.

TABLE I
Example values of the latch period (in microseconds, μs), latching
current (mA) and holding current (mA) that may be selected by control
circuitry of a medical device for the first phase and the second
phase of a biphasic pacing pulse based on different starting pacing
pulse amplitudes (V) of the first phase of the pacing pulse.

| | PACING PULSE PHASE | | | | | | |
|---|---|---|---|---|---|---|---|
| | | | | | | | SECOND |
| PULSE | FIRST PHASE | | | | | | PHASE |
| AMPLITUDE | 10 V | 13 V | 16 V | 20 V | 30 V | >30 V | — |
| LATCH | 120 | 120 | 120 | 120 | OFF | OFF | 120 |
| PERIOD (μs) | | | | | | | |
| LATCHING | 80 | 80 | 80 | 40 | OFF | OFF | 100 |
| CURRENT | | | | | | | |
| (mA) | | | | | | | |
| HOLDING | 40 | 40 | 20 | 10 | OFF | OFF | 40 |
| CURRENT | | | | | | | |
| (mA) | | | | | | | |

As an illustrative example, the pacing pulse amplitude may be 20 V. The pacing pulse amplitude may be programmed by a user or adjusted to 20 V based on a pacing capture test. The high side switches 180a-c may have a specified minimum latch period of 120 microseconds. Control circuit 80 may determine the first phase latching current to be 40 mA and the first phase holding current to be 10 mA. As further described below, the second phase latching current and the second phase holding current may be higher than the latching current and the holding current pulled during the first phase of a biphasic pulse. In this example, the second phase latching current is 100 mA and the second phase holding current is 40 mA when the pacing pulse amplitude (starting amplitude of the first phase) is 20 V. It is to be understood that the latching and holding currents listed in Table I refer to the latching and holding currents that are pulled by the internal adjustable load. The total current flowing through the high side switch is the sum of the adjustable load current and the external pacing load current. This total current meets the required current for maintaining a triggered high side switch 180a, 180b or 180c in a conducting state.

Table I may represent the highest latching and holding currents that the internal adjustable load may be configured to pull because the external pacing load may be expected to be a maximum of about 250 ohms or less. The external pacing load may be in the range of 30 ohms to 250 ohms for example. When the external pacing load impedance is high, the current flow through the pacing load is relatively lower than when the external pacing load impedance is relatively lower. As such, the internal adjustable load may be controlled to pull relatively lower latching current and/or holding current during a given phase of a pacing pulse when the external pacing load impedance is relatively lower, corresponding to a higher current flow through the external pacing load. As such, multiple look up tables may be stored in memory 82 including values of the adjustable load latching and holding currents for different combinations of pacing pulse amplitude and external load impedance. In this way, the latching and holding currents pulled by the adjustable load can be selected so that the total current that is pulled through a high side switch during a given phase of the pacing pulse can be controlled to minimize the likelihood of the high side switch being disabled prematurely while minimizing the current through the internal adjustable load to conserve power source 98 and avoid a rapidly decaying pacing pulse amplitude.

Memory 82 may store a look up table (analogous to Table I above) of latching currents and holding currents for each available pacing pulse amplitude for a given pacing electrode vector (e.g., having an assumed default external pacing load impedance or for multiple different impedances). When more than one pacing electrode vector is available, e.g., programmably selected by a user, a look up table of latching and holding currents for each available pacing pulse amplitude may be stored for each pacing electrode vector having an assumed pacing load impedance. In some cases, the external pacing load impedance associated with each of the available pacing electrode vectors that may be coupled to HV output circuit 160 may be similar. For example, with reference to FIG. 1, the external pacing load impedance between coil electrodes 24 and 26, between coil electrode 24 and housing 15 and between coil electrode 26 and housing 15 may be expected to be similar such that different look up tables for the three different pacing electrode vectors may not be required.

In other examples, control circuit 80 may determine the pacing electrode vector impedance by controlling therapy delivery circuit 84 to apply an impedance measurement drive signal (e.g., a known voltage or current signal) to the pacing electrode vector and receiving a resulting current or voltage signal via sensing circuit 86. In some examples, ICD 14 may include an impedance measurement circuit used for measuring and monitoring various lead impedances, pacing electrode vector impedances and/or CV/DF electrode vector impedance. When control circuit 80 is configured to obtain an impedance measurement, the pacing electrode vector impedance (also referred to herein as the “external load impedance”) may be determined by an impedance measurement for use in determining the first phase latching current 224 and/or first phase holding current 226 based on the external pacing load current determined from the pacing pulse amplitude 204 and the measured pacing electrode vector impedance and a known, specified latching current required for the high side switches. In other examples, when the external pacing load impedance is measured for the selected pacing electrode vector, control circuit 80 may select a look up table stored in memory 82 for a corresponding external pacing load impedance range. Control circuit 80 may look up the latching current and holding current to be pulled by adjustable load 156 for a given pulse amplitude 204 from the selected look up table for each phase of the cardiac pacing pulse.

When the pacing pulse is a biphasic pulse (as shown in FIG. 6) or a multiphasic pulse, control circuit 80 may control adjustable load 156 to draw a latching current and holding current during each phase of the pacing pulse. Referring to the example of FIG. 6, the first high side switch 180a, 180b or 180c that is held in an ON state during the first phase 203 of pacing pulse 202, may be disabled at the expiration of the first phase duration 212a by switching OFF the low side switch 182a, 182b, or 182c (and disabling the internal adjustable load 156), thereby starving the high side switch 180a, 180b or 180c of current needed to remain in a conducting state. The holding current 226 may be terminated by control circuit 80 by controlling adjustable load 156 to be off or disabled such that no internal current is pulled through the high side switch. Control circuit 80 may apply a trigger current 236 to a second high side switch 180a, 180b or 180c (different than the first high side switch) to reverse the polarity of the delivered pacing pulse 202 for the second phase 205. The trigger current 236 turns the second high side switch to an ON state from an OFF state. To avoid excessive current drain due to maintaining the trigger current during the pacing pulse 202 and to avoid premature truncation of pacing pulse 202, adjustable load 156 is controlled by control circuit 80 to pull a second latching current 228 during latch period 225 at the start of the second phase 205 of pacing pulse 202, when trigger current 236 is removed. At the expiration of the latch period 225, adjustable load 156 is controlled by control circuit 80 to pull a second holding current 230 for a holding period 232b that extends from the expiration of the latch period 225 until the expiration of the second phase duration 212b (and the expiration of the pacing pulse width 212).

The second phase latching current 228 can be greater than the first phase latching current 224 in some examples. The second phase holding current 230 can be greater than the first phase holding current 226 in some examples. Control circuit 80 may select the second phase latching current 228 and/or the second phase holding current 230 based on the starting amplitude 208 of the second phase 205, which is opposite in polarity but corresponds to the ending pulse amplitude 206 of the first phase 203. Because the starting amplitude 208 of the second phase 205 is a lower voltage than the starting pacing pulse amplitude 204, the external pacing load current during the second phase 205 of pacing pulse 202 is lower than the external pacing load current during the first phase 203. To account for this lower external pacing load current, the internal adjustable load current pulled during the second phase 205 may be increased by control circuit 202 to avoid premature truncation of the pacing pulse 202 due to insufficient current flow through the high side switch 180a, 180b or 180c for maintaining the high side switch in an ON state.

In some examples, the control circuit 80 may measure the voltage of the holding capacitor(s) being discharged during the first phase 203 of the pacing pulse 202. The pulse voltage amplitude may be sampled at one or more time points during cardiac pacing pulse 202. For example, the ending voltage 206 of the first phase 203 may be sampled and used by control circuit 80 for determining the second phase latching current 228 in combination with a measured or assumed default pacing electrode vector impedance and the known specified latching current required to maintain the high side switch in an ON state during the latch period 225. In other examples, the second phase latching and second phase holding currents 228 and 230 may be determined by control circuit 80 from a look up table stored in memory 82 similar to Table I above for a measured or assumed default pacing electrode vector impedance and based on the starting pacing pulse amplitude 204. The rate of discharge of the holding capacitor(s) discharged from a starting pacing pulse amplitude 204 may be determined or known based on the capacitance of the voltage source and the measured or assumed pacing electrode vector impedance. For example, the RC time constant “tau” for the pacing voltage source and external pacing load impedance may be used to estimate the ending amplitude of the first phase 203 for a given starting pulse amplitude 204. Thus, the ending amplitude 206 and corresponding starting amplitude 208 of the second phase 205 may be known or predictable for a given starting pacing pulse amplitude 204 (of first phase 203). The required second phase latching current 228 and second phase holding current 230 pulled during the second phase 205 of a biphasic pacing pulse 202 may be modeled or determined through bench testing, for example, and may be stored in memory 82 in a look up table for a given pacing electrode vector having an assumed default impedance (or a measured pacing electrode vector impedance) and a programmed pacing pulse amplitude 204.

Table I above provides various examples of first and second latching currents and first and second holding currents that may be drawn by adjustable load 156 during respective first and second phases of the cardiac pacing pulse under the control of control circuit 80 for different pacing pulse amplitudes. The first and second phase latching currents may be selectable from a range of 20 mA to 100 mA in 20 mA steps in an example. For instance, the first and second phase latching currents may be selectively controlled to be 20 mA, 40 mA, 60 mA, 80 mA or 100 mA. The first and second phase holding currents may be selectable from a range of 5 mA to 70 mA in 10 to 15 mA steps in some examples. In one example, the first and second phase holding currents may be selectively controlled to be 10 mA, 20 mA, 30 mA, 40 mA or 50 mA.

The holding capacitor(s) being discharged for generating pacing pulse 202 may discharge at a faster rate when the internal load current 222 is pulled compared to when no internal load current is pulled in addition to the external pacing load current. However, this increased rate in capacitor discharge (and associated increased rate of exponential decay of each phase of the pacing pulse) is expected to be minimal or negligible over the pacing pulse width 212. For instance, for a 175 microfarad capacitance, the additional discharge during the latch period 225 due to pulling an 80 mA latching current may be about 0.055 V (which may be calculated from dV=dt*I/C where dV is the additional discharge caused by the latching current I, C is the capacitance and dt is the latch period, e.g., 120 microseconds). As such, the first phase holding current, the second phase latching current, and the second phase holding current may be selected or determined based on the starting pacing pulse amplitude 204 without necessarily having to account for a change in the capacitor discharge rate and pacing pulse decay rate associated with any of the respectively preceding first phase latching current, first phase holding current or second phase latching current. However, when control circuit 80 is configured to determine the ending amplitude 206 of the first phase 203, any increase or variation in pacing pulse decay rate due to the first phase latching current 224 and/or the first phase holding current 226, among other factors, may be taken into account when control circuit 80 determines the subsequent second phase latching current 228 and/or second phase holding current 230 based on the ending amplitude 206 of first phase 203.

Furthermore, in some examples, control circuit 80 may monitor the pacing pulse voltage amplitude during the first phase 203 and/or second phase 205, e.g., at least at the expiration of the latch periods 225. If the pacing pulse decay rate is faster than expected, e.g., a lower voltage measurement than expected, control circuit 80 may adjust a subsequent first phase holding current 226, second phase latching current 228 and/or second phase holding current 230 as needed based on the monitored voltage amplitude of the pacing pulse 202 to maintain the current flowing through the enabled high side switch as needed to hold the switch in an ON state. Control circuit 80 may monitor the external pacing load impedance to correct for changes in impedance that change the external load current. If the external pacing load current increases or decreases, e.g., due to a change in pacing electrode vector impedance, during or between cardiac pacing pulses, control circuit 80 may make appropriate adjustments to the internal load current pulled by adjustable load 156 during the latch period 225 and/or holding period 232a and/or 232b of the first phase 203 and/or second phase 205 of the pacing pulse.

FIG. 7 is a diagram 300 of a biphasic cardiac pacing pulse 302 that may be delivered via the HV output circuit 160 shown in FIGS. 4 and 5 and a corresponding current signal 322 that may be drawn by the internal adjustable load 156 during the biphasic pacing pulse 302 according to another example. Biphasic pacing pulse 302 includes a first phase 303 having duration 312a and a second phase 305 having duration 312b for a total pacing pulse width 312 as generally described above in conjunction with FIG. 6. However, in this example pacing pulse 302 has a starting pulse amplitude 304 that may be in an upper range of pacing voltage amplitudes, e.g., 30 V or higher.

A trigger current 334 may be applied to a selected high side switch 180a, 180b or 180c by control circuit 80 to turn the switch from an OFF state to an ON state at the expiration 316 of a pacing interval 314. In this case, the current flow through the selected high side switch due to holding capacitor discharge (e.g., HV holding capacitor 162 discharge) starting from the pacing pulse amplitude 304 is sufficient to latch and hold the high side switch in a conducting state during the first phase 303 of pacing pulse 302 when the trigger current 334 is removed. As shown in example Table I above, when the pacing pulse amplitude is 30 V or higher, the internal adjustable load 156 may be off or disabled during the first phase 303 of pacing pulse 302. The first phase latch period may be set to zero or “off,” the first phase latching current may be set to zero or “off,” and the first phase holding current may be set to zero or “off.”

As the holding capacitor(s) of the cardiac pacing voltage source discharge to the ending amplitude 306 of the first phase 303, however, the current flow through the high side switch 180a, 180b, or 180c decreases. In order to prevent premature truncation of the pacing pulse 302 during the second phase 305, control circuit 80 may set the latch period 325 to start upon removal of the trigger current 336 that turns a second switch of high side switches 180a-c from an OFF state to an ON state to switch the polarity between the first phase 303 and the second phase 305 of pacing pulse 302. Control circuit 80 may control the adjustable load 156 to pull a second phase latching current 328 during the latch period 325 and a second phase holding current 330 during a holding period 332 until the expiration of the pacing pulse width 312.

As shown by the examples given in Table I, depending on the starting pacing pulse amplitude, a first phase latching current and a first phase holding current may be selected to maintain a first high side switch 180a, 180b or 180c in an ON state. When the starting pacing pulse amplitude is greater than a specified threshold voltage, the current flow to the external pacing load may be sufficient to maintain the first high side switch in the ON state without pulling additional current by the internal adjustable load 156 during the first phase of the cardiac pacing pulse. The second phase latching current and the second phase holding current may be higher than the first phase latching current and the first phase holding current (which may both be zero), respectively, because the external pacing load current is lower during the second phase 305 of a biphasic pacing pulse 302 due to the exponentially decaying pacing pulse voltage amplitude. Furthermore, as described above, the holding current 330 drawn during a given pacing pulse phase may be lower than the latching current 328 to reduce unnecessary current drain of the ICD power source 98 (shown in FIG. 3). In this way, the internal current drain used for maintaining the high side switches 180a-c in a conducting state can be minimized to enable cardiac pacing pulse delivery via HV output circuit 160 with no or insignificant shortening of the useful life of ICD 14.

In the examples shown in Table I, the second phase latching current and second phase holding current are fixed values. The second phase latching current and the second phase holding current may be selected based on a predictable decay rate of the pacing pulse 302 during the first phase 303. As described above, however, the second phase latching and holding currents may be fine-tuned in some examples by determining the ending voltage 306 of the first phase 303 and/or the pacing voltage amplitude at the end of the second phase latch period 325 and/or by performing one or more pacing electrode vector impedance measurements before and/or during the cardiac pacing pulse delivery.

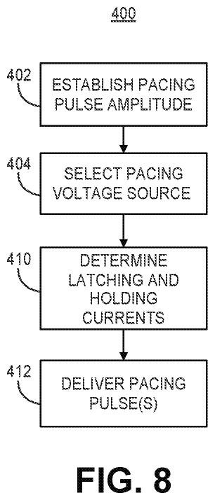

FIG. 8 is a flow chart 400 of a method for delivering cardiac pacing pulses by ICD 14 according to some examples. At block 402, control circuit 80 may establish the pacing pulse amplitude. The pacing pulse amplitude may be established by performing a pacing capture test. The pacing capture test may include delivering one or more pacing pulses at a known pulse energy, e.g., one or more pacing pulse amplitudes for a given pulse width.

The pacing capture test may be performed to confirm myocardial capture occurs a given pulse energy. The pacing capture test may be performed to determine the pacing capture threshold as the lowest pacing pulse amplitude for a given pulse width at which a pacing evoked response (capture) occurs.

In some instances, control circuit 80 may initiate a pacing capture test at block 402 in response to detecting loss of capture or according to a daily or other scheduled pacing capture test or capture management protocol. Control circuit 80 may control therapy delivery circuit 84 to deliver a cardiac pacing pulse at one or more pacing pulse amplitudes. Capture may be verified by detecting an evoked response QRS waveform in a cardiac electrical signal sensed by sensing circuit 86 in some examples.

In some cases, the pacing capture test is performed to determine the pacing capture threshold for at least one pacing electrode vector. A coil-to-coil pacing electrode vector between coil electrodes 24 and 26 or another low impedance pacing electrode vector between housing 15 and one or both of coil electrodes 24 and/or 26 may be used during the pacing capture test in some examples. The internal adjustable load 156 of HV output circuit 160 may be controlled by control circuit 80 as needed during test pacing pulse delivery to pull current according to any of the methods described above, e.g., based on the test pulse amplitude(s). The pacing pulse amplitude may be established at block 402 based on a test pacing pulse amplitude that is determined to result in confirmed cardiac capture. The pacing pulse amplitude may be established based on the determined capture threshold that is the lowest voltage amplitude for a given pulse width that successfully causes myocardial depolarization. The pacing pulse amplitude used by therapy delivery circuit 84 to generate pacing pulses may be established by control circuit 80 at block 402 to be a safety margin (e.g. 0.25 to 5 V or 0.5 to 2 V as examples) greater than a pacing capture threshold.

In some examples, capture test pulses may be delivered using multiple pacing electrode vectors selected from among the available electrodes, e.g., electrodes 24, 26, 28, 30 and housing 15 as shown in FIG. 1. The pacing electrode vector associated with the lowest pacing capture threshold (or a lowest pacing pulse amplitude at which capture is verified) may be identified and selected for delivering cardiac pacing pulses. The pacing pulse amplitude may be established to be a safety margin greater than the pacing capture threshold or other pacing pulse amplitude at which capture is verified.

In other examples, the pacing pulse amplitude is established at block 402 by control circuit 80 based on receipt of a user programmed value via telemetry circuit 88, which may be stored in memory 82. In still other examples, the pacing pulse amplitude may be a default or nominal pacing pulse amplitude that is stored in memory 82.

The cardiac pacing method of flow chart 400 is described for the situation of delivering cardiac pacing pulses using electrode terminals 124, 126 and/or 115, electrically coupled to respective coil electrode 24, coil electrode 26 and housing 15, defining a low impedance external pacing load. In this case, the HV output circuit 160, including high side switches 180a-c, is being used for delivering the pacing pulses, which may require an internal load current for operating the high side switches 180a-c, depending on the pacing pulse amplitude established at block 402. It is to be understood that in some instances, cardiac pacing may be delivered by ICD 14 using a different pacing electrode vector that does not require the use of HV output circuit 160 including high side switches 180a-c. For example, in some instances, ICD 14 may be configured for delivering cardiac pacing by LV therapy circuit 102. The method of flow chart 400, however, is performed in conjunction with pacing pulse delivery via HV output circuit 160.

If multiple pacing voltage sources are available, e.g., as described in conjunction with FIG. 5, control circuit 80 may select a cardiac pacing voltage source at block 404 based on the pacing pulse amplitude established at block 402. For example, control circuit 80 may compare the established pacing pulse amplitude to at least a lower range and an upper range of pacing voltage amplitudes. In some examples, an intermediate range of pacing voltage amplitudes may be available from a cardiac pacing voltage source. The lower, optional intermediate, and upper ranges of pacing pulse amplitudes may be predefined and stored in memory 82. The lower, intermediate, and upper ranges can correspond to the maximum pacing pulse amplitude available from a given cardiac pacing voltage source. For example, with reference to FIG. 5, LV charging circuit 132 may be capable of charging a LV holding capacitor 142 or 146 for delivering a cardiac pacing pulse signal in the lower range, e.g., up to a maximum of 8 to 10 V, which may include composite pacing pulses as generally disclosed in the above-incorporated U.S. Pat. No. 10,449,362 (Anderson, et al.). In some examples, LV charging circuit 132 may include multiple charge pumps to enable charging of a LV holding capacitor 142 or 146 (or a combination of both) to higher voltages, e.g., 16 to 20 V, as the maximum available voltage amplitude for the lower range.

HV charging circuit 152 may charge HV holding capacitor 162 to an intermediate voltage to enable voltage regulator 154 to pass a cardiac pacing voltage signal to HV output circuit 160 having an amplitude in an intermediate range, e.g., greater than the maximum limit of the lower range (maximum voltage available from LV therapy circuit 102) and up to 16 V, up to 20 V, up to 30 V or up to 40 V in various examples. HV charging circuit 152 charging HV capacitor 162 may be capable of generating cardiac pacing pulses in an upper range, above the maximum limit of the output of voltage regulator 154 and LV therapy circuit 102, e.g., greater than 20 V, greater than 30 V or greater than 40 V. Other examples of pacing amplitude ranges and associated cardiac pacing voltage sources that may be selectable by control circuit 80 are described above, e.g., in conjunction with FIG. 5.

Accordingly, control circuit 80 may select the cardiac pacing voltage source to be received from LV therapy circuit 102 for a pacing pulse amplitude in the lower range, from voltage regulator 154 (utilizing HV charging circuit 152 and HV holding capacitor 162) when the pacing pulse amplitude is in an intermediate range, or from HV charging circuit 152 and HV capacitor 162 when the pacing pulse amplitude is in the upper range. The cardiac pacing voltage sources of therapy delivery circuit 84 may include multiple, selectable cardiac pacing voltage sources capable of generating pacing pulses in different pacing pulse amplitude ranges. In other examples, a default cardiac pacing voltage source, e.g., HV capacitor 162 charged by HV charging circuit 152, may be used having a range of programmable pacing pulse amplitudes including the pacing pulse amplitude that is established at block 402. It is to be understood, therefore, that block 404 may be omitted in some examples when a single pacing voltage source is being used for generating pacing pulses.

At block 410, control circuit 80 may determine the latching current and holding current for each phase of the cardiac pacing pulse based on at least the pacing pulse amplitude established at block 402. Control circuit 80 may determine the latching current and holding current for each phase using any of the techniques described above. In some examples, the latching current and holding current is determined from a look up table stored in memory 82 for each pacing pulse phase based on the pacing pulse amplitude. The latching and/or holding current can be selected from a look up table by control circuit 80 based on an amplitude of the pacing pulse (established at block 402). The latching and/or holding current can be selected from a look up table stored in memory 82 corresponding to the pacing electrode vector impedance, which may be a measured impedance or an estimated default impedance. The latching and holding currents may generally be determined as the difference between a computed or estimated external pacing load current and a known, specified current required for maintaining the high side switches 180a-c in an ON state during the latching period and after the latching period, respectively.

As described above, the holding current for a given pacing pulse phase can be less than the latching current for that pacing pulse phase. When the cardiac pacing pulse is a multiphasic pulse, the first phase may have a lower latching current and lower holding current than subsequent pacing pulse phases and may be zero in some instances. When the established pacing pulse amplitude is greater than a threshold voltage, e.g., 30 V or higher or 40 V or higher, which results in sufficient external pacing load current flowing through the high side switch 180a, 180b or 180c to maintain it in an ON state, the latching and holding currents may be zero in the first phase of the pacing pulse. In a multi-phasic pacing pulse, the latching current and the holding current of each successive phase may be successively increased as the external load current decreases due to the decaying pacing pulse amplitude to avoid premature truncation of the pacing pulse.

After determining the latching and holding currents at block 410, therapy delivery circuit 84 delivers one or more cardiac pacing pulses at block 412, under the control of control circuit 80. Therapy delivery circuit 84 delivers each pacing pulse according to the pacing pulse amplitude established at block 402, having a specified pacing pulse width and number of pacing pulse phases. At the start of each pacing pulse phase, a trigger current can be applied to turn ON a high side switch 180a, 180b, or 180c that is coupled to the respective electrode terminal 124, 126 or 115 electrically connected to the cathode electrode for the given phase. If the latching current is non-zero for the given phase, the latch period may be started upon (or just before) removal of the trigger current and the latching current is drawn by the internal adjustable load 156 for the latch period, e.g., from the start of the pacing pulse phase until the latch period expires. Upon expiration of the latch period, the internal adjustable load may be controlled to pull the lower holding current for the remaining portion of the pacing pulse phase, e.g., from the expiration of the latch period until the expiration of the pacing pulse phase.

Pacing pulses may be delivered at block 412 according to a programmed pacing therapy, e.g., bradycardia pacing, post-shock pacing, ATP, long pause prevention pacing, or any other pacing therapy ICD 14 is configured to deliver. It is recognized that the pacing pulse amplitude may be adjusted from time to time due to capture management protocols or reprogramming of the pacing control parameters, in which case, the control circuit 80 may re-determine the appropriate latching and holding currents as needed for each phase of the cardiac pacing pulses. In some instance, the pacing load impedance may change, e.g., as determined during a lead impedance measurement. Accordingly, it is to be understood that portions of the cardiac pacing method of flow chart 400 may be repeated as needed to make adjustments to the starting pacing pulse amplitude and/or latching and holding currents used in generating and delivering pacing pulses to promote reliable capture of the myocardial tissue in response to the delivered pacing pulses.

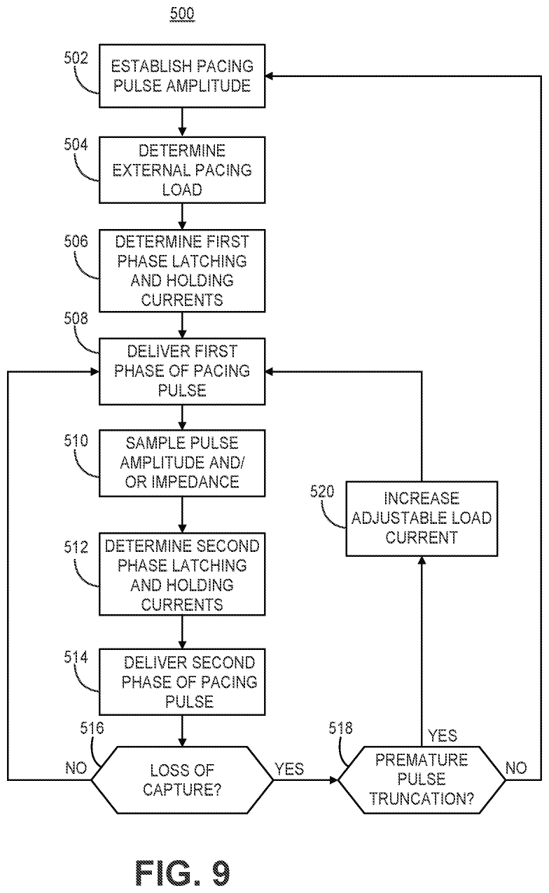

FIG. 9 is a flow chart 500 of a method for delivering cardiac pacing pulses by ICD 14 via the HV output circuit 160 using the internal adjustable load 156 according to another example. At block 502, control circuit 80 may establish the pacing pulse amplitude according to any of the examples given above. The method of flow chart 500 may be performed using a selected cardiac pacing voltage source having a capacitance high enough to deliver sufficient energy to a low impedance pacing electrode vector without excessive decay of the pacing pulse voltage amplitude to less than the pacing capture threshold prior to the expiration of the pacing pulse width. For the sake of convenience, flow chart 500 is described in conjunction with FIG. 4, where the pacing voltage source is the HV capacitor 162 that is chargeable to a shock voltage amplitude but can be charged to the established pacing pulse amplitude by HV charging circuit 152. It is contemplated, however, that a different pacing voltage source may be available as described above, e.g., in conjunction with FIG. 5.

At block 504, control circuit 80 may determine the external pacing load impedance. The external pacing load impedance may be an assumed or predicted impedance based on the selected low impedance pacing electrode vector. The external pacing load impedance may be assumed to be a maximum expected pacing load impedance such that the external pacing load current is anticipated to be relatively low. In this case, the internal adjustable load 156 can be controlled to pull a relatively high current for this “worst case” external pacing load impedance condition to prevent the high side switches 180a-c from turning OFF. In other examples, the external pacing load impedance may be assumed or predicted to be an intermediate impedance in an expected range of pacing load impedances for the selected low impedance pacing electrode vector. In still other examples, control circuit 80 may perform an impedance measurement at block 504 for measuring the actual external pacing load impedance as generally described above in conjunction with FIG. 6.

At block 506, control circuit 80 may determine the latching current and holding current to be pulled by internal adjustable load 156 during the first phase of a cardiac pacing pulse. The pacing pulse may be a monophasic pacing pulse in some examples such that there is only one phase. However, for the sake of example, flow chart 500 is described assuming that the pacing pulse is a biphasic pacing pulse. In other examples, the pacing pulse may include more than two phases, e.g., a triphasic or other multiphasic pacing pulse.

The latching current and holding current determined at block 506 may be determined from a look up table stored in memory 82 corresponding to the external pacing load determined at block 504. Control circuit 80 may fetch the value of the first phase latching current and holding current from the look up table for the pacing pulse amplitude established at block 502. In other examples, control circuit 80 may be configured to compute the first phase latching current and first phase holding current based on the difference between the total specified current required for holding the high side switches 180a-c in a conducting state during and after the latch period, respectively, and the estimated external pacing load current computed from the determined external pacing load and established pacing pulse amplitude.

At block 508, therapy delivery circuit 84 may be controlled by control circuit 80 to deliver the first phase of the pacing pulse having the established pacing pulse amplitude and a specified (e.g., programmed) phase duration. The internal adjustable load 156 is controlled to pull the determined first phase latching and holding currents during a latch period and after the latch period of the first phase duration, respectively. As described above in conjunction with FIG. 7, the first phase latching and holding currents may be zero in some examples.

In some examples, control circuit 80 may be configured to sample the pacing pulse voltage amplitude during pacing pulse delivery and/or monitor the pacing electrode vector impedance during or between pacing pulse delivery. For example, control circuit 80 may determine the ending voltage amplitude of the first phase of the pacing pulse at block 510. In some examples, control circuit 80 may determine an external pacing load impedance based on the ending voltage amplitude. In other examples, the voltage amplitude of the pacing pulse may be sampled during the first phase duration earlier than the end of the first phase. Based on the sampled pacing pulse voltage amplitude and/or pacing load impedance, control circuit 80 may determine the second phase latching and/or holding currents at block 512. The second phase latching and/or holding currents may be determined by control circuit 80 from a look up table stored in memory corresponding to the sampled external load impedance determined at block 510 or the pacing load impedance determined at block 504. The second phase latching and/or holding current may be fetched from the look up table for the pacing pulse voltage amplitude sampled during the first phase of the pacing pulse at block 510, which may be at the expiration of the first phase (corresponding to the starting amplitude of the second phase) or earlier than the expiration of the first phase. When the voltage amplitude is sampled earlier than the expiration of the first phase, the decay rate of the first phase of the pacing pulse may be computed or estimated so that the ending amplitude of the first phase and the starting amplitude of the second phase may be computed based on the estimated decay rate and the time remaining in the first phase of the pacing pulse from the sampled voltage amplitude.

In other examples, control circuit 80 may determine the second phase latching and/or holding currents by determining the difference between the total specified current required to hold the high side switches 180a-c in a conducting state during and after the latch period, respectively, and the estimated external pacing load current computed from the external pacing load (sampled at block 510 or determined at block 504) and the sampled pacing pulse amplitude. In this way, control circuit 80 may determine the second phase latching and holding currents based on the decay behavior of the first phase of the pacing pulse and the expected external pacing load current during the second phase of the pacing pulse.

At block 514, therapy delivery circuit 84 is controlled by control circuit 80 to deliver the second phase of the pacing pulse. As described above, e.g., in conjunction with FIG. 4, a high side switch 180a, 180b, or 180c coupled to the pacing electrode cathode during the first phase can be disabled by turning OFF the low side switch 182a, 182b or 182c coupled to the pacing electrode anode (and terminating any first phase holding current being pulled by internal adjustable load 156) to starve the high side switch of the required current to maintain a conducting state. The polarity of the pacing pulse can be reversed between the first phase and the second phase by applying a trigger current to a different high side switch 180a, 180b or 180c coupled to the pacing electrode anode (during the first phase and now the pacing electrode cathode during the second phase) and turning ON the low side switch 182a, 182b or 182c coupled to the pacing electrode cathode (during the first phase and now the pacing electrode anode during the second phase).

The internal adjustable load is controlled to pull the second phase latching current and holding current determined at block 512 during the latch period and after the latch period, respectively, as generally described above in conjunction with FIGS. 6 and 7. The pacing pulse can be terminated at the expiration of the second phase duration (and the expiration of the pacing pulse width) by turning OFF the low side switch 182a, 182b or 182c and terminating the holding current being pulled by the internal adjustable load 156.

In some examples, control circuit 80 may be configured to determine when an adjustment to the latching and/or holding current for one or more phases of the cardiac pacing pulse is needed. For instance, if the pacing pulse amplitude is decaying faster than expected during a first phase of the biphasic pacing pulse, the latching and/or holding current during a second phase of the pacing pulse may need to be increased. In other examples, if the pacing electrode vector impedance changes from one measurement to the next, e.g., between daily pacing electrode vector impedance measurements, control circuit 80 may re-determine the latching and holding currents for each phase of the cardiac pacing pulses.

In some examples, control circuit 80 may monitor for loss of capture (block 516) and/or premature truncation of the pacing pulse (block 518). If loss of capture is detected at block 516, e.g., based on no evoked response detected following the pacing pulse, the pacing capture threshold may have increased or the pacing pulse may be truncated prematurely due to insufficient current flow through the high side switch prior to expiration of the pacing pulse width. Control circuit 80 may be configured to detect or determine likely premature truncation of a pacing pulse and may control the internal adjustable load 156 to increase the latching and/or holding current (block 520) for one or more phases of the pacing pulse.

If loss of capture is not detected at block 516, control circuit 80 may return to block 508 and continue to deliver cardiac pacing pulses as needed according to a pacing therapy without adjusting the internal adjustable load currents. If loss of capture is detected at block 516, control circuit 80 may determine if the loss of capture is due to premature pulse truncation at block 518. Premature pulse truncation may be determined based on sampling the pacing pulse voltage amplitude during the first phase and/or the second phase of the pacing pulse. If the pacing pulse voltage amplitude delivered to the electrode terminal drops to zero, the high side switch 180a, 180b or 180c may have turned OFF prematurely due to insufficient current flow. In some examples, monitoring for an evoked response to detect loss of capture may be optional at block 516. Control circuit 80 may monitor for premature pulse truncation at block 518 without necessarily monitoring for loss of capture at block 516.

In response to detecting premature pulse truncation (or evidence of premature pulse truncation based on detecting loss of capture), control circuit 80 may increase the adjustable load current at block 520. The first phase latching and/or holding current may be increased and/or the second phase latching and/or holding current may be increased at block 520. The first phase latching current may be increased when control circuit 80 detects premature pulse truncation during the first phase latch period. The first phase holding current may be increased when control circuit 80 detects premature pulse truncation during the first phase but after the first phase latch period. The second phase latching current and/or holding current may be increased when control circuit 80 detects premature pulse truncation during the latch period or after the latch period of the second phase of the pacing pulse. The adjustable load current may be increased by a specified increment or up to a maximum latching or holding current, which may be equal to the total specified current required for holding the high side switch in a conducting state during the latching period or after the latching period, respectively. The adjustable load current may be increased up to a maximum latching or holding current, which may be equal to the total specified current required for holding the high side switch in a conducting state during or after the latching period, respectively, less an assumed minimum external load current. Control circuit 80 may return to block 508 to deliver the next cardiac pacing pulse according to the increased adjustable load current(s). In some instances, the pacing pulse amplitude may be increased in addition to or alternatively to increasing the adjustable load current during a subsequent pacing pulse in response to determining premature truncation of a delivered pacing pulse.

In the example shown, when premature pulse truncation is not detected at block 518, but loss of capture has been detected at block 516, control circuit 80 may return to block 502 to re-establish the pacing pulse amplitude. The pacing capture threshold may have increased. Control circuit 80 may perform a pacing capture test to determine a new pacing pulse amplitude and subsequently redetermine the first phase latching and holding currents (block 506) based on the updated pacing pulse amplitude for use in delivering the next pacing pulse. In other examples, if premature pacing pulse truncation is being monitored without necessarily determining if loss of capture has occurred or not following each pacing pulse (e.g., block 516 omitted), control circuit 80 may return to block 508 when premature pulse truncation is not detected at block 518, without adjusting the internal adjustable load current.

Instead of performing loss of capture monitoring following each pacing pulse at block 516, control circuit 80 may be configured to perform a scheduled pacing capture test and/or pacing electrode vector impedance measurement according to a capture management and/or impedance monitoring protocol, e.g., once per day during the night or other scheduled basis. In this case, control circuit 80 may repeat the process of flow chart 500 when a change in pacing capture threshold and/or change in pacing electrode vector impedance is determined. It is to be understood that detection of loss of capture may be an indication of premature truncation such that premature truncation of the pacing pulse may be inferentially determined from a loss of capture detection without necessarily detection premature truncation directly. It is to be understood that detection of premature truncation may be an indication of loss of capture such that loss of capture of the pacing pulse may be inferentially determined from detecting premature truncation of a delivered pacing pulse without necessarily detecting loss of capture (e.g., absence of a pacing evoked response) directly. Thus in some examples, control circuit 80 may monitor loss of capture, premature truncation, or both following a given pacing pulse.

Further disclosed herein is the subject matter of the following examples:

Example 1. A medical device including a therapy delivery circuit configured to deliver electrical stimulation pulses comprising and control circuitry. The therapy delivery circuit including a first electrode terminal, a second electrode terminal, a high voltage output circuit comprising a first high side switch coupled to the first electrode terminal, an internal adjustable load coupled to a low side of the first high side switch; and a cardiac pacing voltage source configured to generate a first cardiac pacing pulse having a pacing pulse amplitude. The control circuitry being configured to control the therapy delivery circuit to deliver the first cardiac pacing pulse via the first electrode terminal and the second electrode terminal by controlling the internal adjustable load to pull a first latching current to hold the first high side switch in a conducting state during a first portion of the first cardiac pacing pulse and controlling the internal adjustable load to pull a first holding current to hold the first high side switch in a conducting state during a second portion of the first cardiac pacing pulse, the first holding current being less than the first latching current.

Example 2. The medical device of example 1 wherein the control circuitry is further configured to determine the first latching current based on at least the pacing pulse amplitude.

Example 3. The medical device of any of examples 1-2 wherein the therapy delivery circuit further includes a second high side switch coupled to the second electrode terminal and the control circuitry being further configured to disable the second high side switch coupled to the second electrode terminal and enable the first high side switch coupled to the first electrode terminal to reverse a polarity of the first cardiac pacing pulse between a first phase and a second phase of the first cardiac pacing pulse, the second phase comprising the first portion and the second portion of the first cardiac pacing pulse. The control circuit being further configured to control the internal adjustable load to pull a second latching current to hold the second high side switch in a conducting state during a latch period of a first phase of the first cardiac pacing pulse and control the internal adjustable load to pull a second holding current to hold the second high side switch in a conducting state after the latch period of the first phase of the first cardiac pacing pulse, the second holding current being less than the second latching current.

Example 4. The medical device of example 3 wherein the control circuitry is further configured to control the internal adjustable load to pull the second latching current during the first phase of the first cardiac pacing pulse by pulling a lower current than the first latching current pulled during the second phase of the first cardiac pacing pulse.

Example 5. The medical device of any of examples 3-4 wherein the control circuitry is further configured to control the internal adjustable load to pull the second holding current during the first phase of the first cardiac pacing pulse by pulling a lower current than the first holding current pulled during the second phase of the first cardiac pacing pulse.

Example 6. The medical device of any of examples 3-5 wherein the control circuitry is further configured to sample a voltage amplitude of the first cardiac pacing pulse and determine the first latching current pulled during the second phase of the first cardiac pacing pulse based on the sampled voltage amplitude.

Example 7. The medical device of any of examples 1-6 wherein the cardiac pacing voltage source is further configured to generate a second pacing pulse having the pacing pulse amplitude. The control circuitry is further configured to determine an early truncation of a phase of the first cardiac pacing pulse and control the internal adjustable load to pull at least one of an increased latching current or an increased holding current during the second cardiac pacing pulse in response to determining the early truncation.

Example 8. The medical device of any of examples 1-7 wherein the control circuitry is further configured to select the first latching current based on a pacing load impedance coupled to the first electrode terminal and the second electrode terminal.

Example 9. The medical device of any of examples 1-2 wherein the therapy delivery circuit further includes a second high side switch coupled to the second electrode terminal. The control circuitry being further configured to disable the second high side switch coupled to the second electrode terminal and enable the first high side switch coupled to the first electrode terminal to reverse a polarity of the first cardiac pacing pulse between a first phase and a second phase of the first cardiac pacing pulse, the second phase comprising the first portion and the second portion of the first cardiac pacing pulse. The control circuit being further configured to disable the internal adjustable load during the first phase of the first cardiac pacing pulse to pull zero current during the first phase.

Example 10. The medical device of any of examples 1-9 wherein the therapy delivery circuit further comprises a high voltage charging circuit and a high voltage capacitor chargeable to a shock voltage amplitude for delivering cardioversion/defibrillation shocks via the high voltage output circuit. The cardiac pacing voltage source comprising the high voltage capacitor charged by the high voltage charging circuit to a voltage that is less than the shock voltage amplitude.

Example 11. The medical device of any of examples 1-9 wherein the cardiac pacing voltage source comprises a first pacing voltage source configured to generate cardiac pacing pulses in a first range of voltage amplitudes, a second pacing voltage source configured to generate cardiac pacing pulses in a second range of voltage amplitudes, the second range of voltage amplitudes greater than the first range of voltage amplitudes. The control circuitry being further configured to select the cardiac pacing voltage source from the first pacing voltage source and the second pacing voltage source based on the pacing pulse amplitude.

Example 12. The medical device of any of examples 1-11 wherein the control circuitry is further configured to establish the pacing pulse amplitude by controlling the therapy delivery circuit to perform a pacing capture test.

Example 13. The medical device of any of examples 1-12 further comprising a memory storing a lookup table of values of the first latching current and the first holding current for each of a plurality of pacing voltage amplitudes comprising the pacing pulse amplitude. The control circuitry being configured to determine the first latching current and the second latching current from the lookup table based on the pacing pulse amplitude.

Example 14. The medical device of any of examples 1-13 wherein the first electrode terminal is couplable to a cardioversion/defibrillation electrode and the second terminal is couplable to a second cardioversion/defibrillation electrode, at least one of the first cardioversion/defibrillation electrode and second cardioversion/defibrillation electrode carried by an extra-cardiac lead for delivery of the first cardiac pacing pulse and for delivery of cardioversion/defibrillation shock pulses by the therapy delivery circuit.

Example 15. The medical device of any of examples 1-14 wherein the high voltage output circuit further comprises a low side switch coupled to the second electrode terminal. The control circuitry being further configured to disable the first high side switch by turning off the low side switch at an expiration of a phase duration of the first cardiac pacing pulse.

Example 16. A method including generating a first cardiac pacing pulse having a pacing pulse amplitude for delivery via a first electrode terminal and a second electrode terminal of a medical device, pulling a first latching current by an internal adjustable load of the medical device coupled to a low side of a first high side switch coupled to the first electrode terminal to hold the first high side switch in a conducting state during a first portion of the first cardiac pacing pulse, and pulling a first holding current to hold the first high side switch in a conducting state during a second portion of the first cardiac pacing pulse, the first holding current being less than the first latching current.

Example 17. The method of example 16 further comprising determining the first latching current based on at least the pacing pulse amplitude.

Example 18. The method of any of examples 16-17 further comprising disabling a second high side switch coupled to the second electrode terminal and enabling the first high side switch coupled to the first electrode terminal for reversing a polarity of the first cardiac pacing pulse from a first phase to a second phase, the second phase comprising the first portion and the second portion of the first cardiac pacing pulse. The method further including pulling a second latching current by the internal adjustable load to hold the second high side switch in a conducting state during a latch period of the first phase of the first cardiac pacing pulse and pulling a second holding current to hold the second high side switch in a conducting state after the latch period of the first phase of the first cardiac pacing pulse, the second holding current being less than the second latching current.

Example 19. The method of example 18 further comprising pulling the second latching current during the first phase of the first cardiac pacing pulse by pulling a lower current than the first latching current pulled during the second phase of the first cardiac pacing pulse.

Example 20. The method of any of examples 18-19 further comprising pulling the second holding current during the first phase of the first cardiac pacing pulse by pulling a lower current than the first holding current pulled during the second phase of the first cardiac pacing pulse.

Example 21. The method of any of examples 18-20 further comprising sampling a voltage amplitude of the first cardiac pacing pulse and determining the first latching current pulled during the second phase of the first cardiac pacing pulse based on the sampled voltage amplitude.

Example 22. The method of any of examples 16-20 further comprising determining an early truncation of a phase of the first cardiac pacing pulse, generating a second pacing pulse having the pacing pulse amplitude and pulling at least one of an increased latching current or an increased holding current during the second cardiac pacing pulse in response to determining the early truncation.

Example 23. The method of any of examples 16-22 further comprising selecting the first latching current based on a pacing load impedance coupled to the first electrode terminal and the second electrode terminal.

Example 24. The method of any of examples 16-17 further comprising disabling a second high side switch coupled to the second electrode terminal and enabling the first high side coupled to the first electrode terminal to reverse a polarity of the first cardiac pacing pulse between a first phase and a second phase of the first cardiac pacing pulse, the second phase comprising the first portion of the first cardiac pacing pulse and the second portion of the first cardiac pacing pulse. The method further including disabling the internal adjustable load during the first phase of the first cardiac pacing pulse to pull zero current during the first phase.

Example 25. The method of any of examples 16-24 wherein generating the cardiac pacing pulse comprises charging a high voltage capacitor to a voltage less than a shock voltage amplitude, the high voltage capacitor being chargeable to a shock voltage amplitude for delivering cardioversion/defibrillation shocks.

Example 26. The method of any of examples 16-24 further comprising selecting, based on the pacing pulse amplitude, a cardiac pacing voltage source for generating the first cardiac pacing pulse from at least a first pacing voltage source configured to generate cardiac pacing pulses in a first range of voltage amplitudes and a second pacing voltage source configured to generate cardiac pacing pulses in a second range of voltage amplitudes.

Example 27. The method of any of examples 16-26 further comprising performing a pacing capture test to establish the pacing pulse amplitude.

Example 28. The method of any of examples 16-27 further comprising storing a lookup table of values of the first latching current and the first holding current for each of a plurality of pacing voltage amplitudes comprising the pacing pulse amplitude and determining the first latching current and the second latching current from the lookup table based on the pacing pulse amplitude.

Example 29. The method of any of examples 16-28 further comprising delivering the first cardiac pacing pulse when the first electrode terminal is coupled to a first cardioversion/defibrillation electrode and the second terminal is coupled to a second cardioversion/defibrillation electrode, the first and second cardioversion/defibrillation electrodes carried by an extra-cardiac lead for delivery of the first cardiac pacing pulse and for delivery of cardioversion/defibrillation shock pulses.

Example 30. The method of any of examples 16-29 further comprising disabling the first high side switch by turning off a low side switch coupled to the second electrode terminal at an expiration of a phase duration of the first cardiac pacing pulse.

Example 31. A non-transitory, computer readable medium storing a set of instructions that, when executed by control circuitry of a medical device, cause the medical device to generate a cardiac pacing pulse having a pacing pulse amplitude for delivery to an electrode terminal of the medical device, pull a latching current by an internal adjustable load of the medical device coupled to a low side of a high side switch of a high voltage output circuit of the medical device to hold the high side switch in a conducting state during a first portion of the cardiac pacing pulse, and pull a holding current to hold the high side switch in a conducting state during a second portion of the cardiac pacing pulse, the holding current being less than the latching current.

It should be understood that, depending on the example, certain acts or events of any of the methods described herein can be performed in a different sequence, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially. In addition, while certain aspects of this disclosure are described as being performed by a single circuit or unit for purposes of clarity, it should be understood that the techniques of this disclosure may be performed by a combination of units or circuits associated with, for example, a medical device.

In one or more examples, the functions described may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPLAs), or other equivalent integrated or discrete logic circuitry. Accordingly, the term “processor,” as used herein may refer to any of the foregoing structure or any other structure suitable for implementation of the techniques described herein. Also, the techniques could be fully implemented in one or more circuits or logic elements.

Thus, a medical device has been presented in the foregoing description with reference to specific examples. It is to be understood that various aspects disclosed herein may be combined in different combinations than the specific combinations presented in the accompanying drawings. It is appreciated that various modifications to the referenced examples may be made without departing from the scope of the disclosure and the following claims.

1. A medical device comprising:
a therapy delivery circuit configured to deliver electrical stimulation pulses comprising:
a first electrode terminal;
a second electrode terminal;
a high voltage output circuit comprising a first high side switch coupled to the first electrode terminal;
an internal adjustable load coupled to a low side of the first high side switch; and
a cardiac pacing voltage source configured to generate a first cardiac pacing pulse having a pacing pulse amplitude; and
control circuitry configured to control the therapy delivery circuit to deliver the first cardiac pacing pulse via the first electrode terminal and the second electrode terminal by:
controlling the internal adjustable load to pull a first latching current to hold the first high side switch in a conducting state during a first portion of the first cardiac pacing pulse delivered from the cardiac pacing voltage source to the first electrode terminal; and
controlling the internal adjustable load to pull a first holding current to hold the first high side switch in a conducting state during a second portion of the first cardiac pacing pulse, the first holding current being less than the first latching current.

2. The medical device of claim 1 wherein the control circuitry is configured to determine the first latching current based on at least the pacing pulse amplitude.

3. The medical device of claim 1 wherein:
the therapy delivery circuit further includes a second high side switch coupled to the second electrode terminal;
the control circuitry being further configured to:
disable the second high side switch coupled to the second electrode terminal and enable the first high side switch coupled to the first electrode terminal to reverse a polarity of the first cardiac pacing pulse between a first phase and a second phase of the first cardiac pacing pulse, the second phase comprising the first portion and the second portion of the first cardiac pacing pulse;
control the internal adjustable load to pull a second latching current to hold the second high side switch in a conducting state during a latch period of a first phase of the first cardiac pacing pulse; and
control the internal adjustable load to pull a second holding current to hold the second high side switch in a conducting state after the latch period of the first phase of the first cardiac pacing pulse, the second holding current being less than the second latching current.

4. The medical device of claim 3 wherein the control circuitry is further configured to control the internal adjustable load to pull the second latching current during the first phase of the first cardiac pacing pulse by pulling a lower current than the first latching current pulled during the second phase of the first cardiac pacing pulse.

5. The medical device of claim 3 wherein the control circuitry is further configured to control the internal adjustable load to pull the second holding current during the first phase of the first cardiac pacing pulse by pulling a lower current than the first holding current pulled during the second phase of the first cardiac pacing pulse.

6. The medical device of claim 3 wherein the control circuitry is further configured to:
sample a voltage amplitude of the first cardiac pacing pulse; and
determine the first latching current pulled during the second phase of the first cardiac pacing pulse based on the sampled voltage amplitude.

7. The medical device of claim 1 wherein:
the cardiac pacing voltage source is further configured to generate a second pacing pulse having the pacing pulse amplitude;
the control circuitry is further configured to:
determine an early truncation of a phase of the first cardiac pacing pulse; and
control the internal adjustable load to pull at least one of an increased latching current or an increased holding current during the second cardiac pacing pulse in response to determining the early truncation.

8. The medical device of claim 1 wherein the control circuitry is further configured to select the first latching current based on a pacing load impedance coupled to the first electrode terminal and the second electrode terminal.

9. The medical device of claim 1 wherein:
the therapy delivery circuit further includes a second high side switch coupled to the second electrode terminal;
the control circuitry being further configured to:
disable the second high side switch coupled to the second electrode terminal and enable the first high side switch coupled to the first electrode terminal to reverse a polarity of the first cardiac pacing pulse between a first phase and a second phase of the first cardiac pacing pulse, the second phase comprising the first portion and the second portion of the first cardiac pacing pulse; and
disable the internal adjustable load during the first phase of the first cardiac pacing pulse to pull zero current during the first phase.

10. The medical device of claim 1 wherein the therapy delivery circuit further comprises:
a high voltage charging circuit; and
a high voltage capacitor chargeable to a shock voltage amplitude for delivering cardioversion/defibrillation shocks via the high voltage output circuit; and
the cardiac pacing voltage source comprises the high voltage capacitor charged by the high voltage charging circuit to a voltage that is less than the shock voltage amplitude.

11. The medical device of claim 1 wherein:
the cardiac pacing voltage source comprises:
a first pacing voltage source configured to generate cardiac pacing pulses in a first range of voltage amplitudes;
a second pacing voltage source configured to generate cardiac pacing pulses in a second range of voltage amplitudes, the second range of voltage amplitudes greater than the first range of voltage amplitudes;
the control circuitry being further configured to select the cardiac pacing voltage source from the first pacing voltage source and the second pacing voltage source based on the pacing pulse amplitude.

12. The medical device of claim 1 wherein the control circuitry is further configured to establish the pacing pulse amplitude by controlling the therapy delivery circuit to perform a pacing capture test.

13. The medical device of claim 1 further comprising a memory storing a lookup table of values of the first latching current and the first holding current for each of a plurality of pacing voltage amplitudes comprising the pacing pulse amplitude;
wherein the control circuitry is configured to determine the first latching current and the second latching current from the lookup table based on the pacing pulse amplitude.

14. The medical device of claim 1 wherein the first electrode terminal is couplable to a cardioversion/defibrillation electrode and the second terminal is couplable to a second cardioversion/defibrillation electrode, at least one of the first cardioversion/defibrillation electrode and second cardioversion/defibrillation electrode carried by an extra-cardiac lead for delivery of the first cardiac pacing pulse and for delivery of cardioversion/defibrillation shock pulses by the therapy delivery circuit.

15. The medical device of claim 1 wherein:
the high voltage output circuit further comprises a low side switch coupled to the second electrode terminal,
the control circuitry being further configured to disable the first high side switch by turning off the low side switch at an expiration of a phase duration of the first cardiac pacing pulse.

16. A method comprising:
generating a first cardiac pacing pulse having a pacing pulse amplitude for delivery via a first electrode terminal and a second electrode terminal of a medical device;
pulling a first latching current by an internal adjustable load of the medical device coupled to a low side of a first high side switch coupled to the first electrode terminal to hold the first high side switch in a conducting state during a first portion of the first cardiac pacing pulse; and
pulling a first holding current to hold the first high side switch in a conducting state during a second portion of the first cardiac pacing pulse, the first holding current being less than the first latching current.

17. The method of claim 16 further comprising determining the first latching current based on at least the pacing pulse amplitude.

18. The method of claim 16 further comprising:
disabling a second high side switch coupled to the second electrode terminal and enabling the first high side switch coupled to the first electrode terminal for reversing a polarity of the first cardiac pacing pulse from a first phase to a second phase, the second phase comprising the first portion and the second portion of the first cardiac pacing pulse;
pulling a second latching current by the internal adjustable load to hold the second high side switch in a conducting state during a latch period of the first phase of the first cardiac pacing pulse; and
pulling a second holding current to hold the second high side switch in a conducting state after the latch period of the first phase of the first cardiac pacing pulse, the second holding current being less than the second latching current.

19. The method of claim 16 further comprising:
determining an early truncation of a phase of the first cardiac pacing pulse;
generating a second pacing pulse having the pacing pulse amplitude; and
pulling at least one of an increased latching current or an increased holding current during the second cardiac pacing pulse in response to determining the early truncation.

20. A non-transitory, computer readable medium storing a set of instructions that, when executed by control circuitry of a medical device, cause the medical device to:
generate a cardiac pacing pulse having a pacing pulse amplitude for delivery to an electrode terminal of the medical device;
pull a latching current by an internal adjustable load of the medical device coupled to a low side of a high side switch of a high voltage output circuit of the medical device to hold the high side switch in a conducting state during a first portion of the cardiac pacing pulse; and
pull a holding current to hold the high side switch in a conducting state during a second portion of the cardiac pacing pulse, the holding current being less than the latching current.
